SEQUENCE LISTING (2 sequences)

SEQ ID NO: 1 - protein, 2005 aa, Homo sapiens
  REGION 1..2005: note = misc_feature - Homo sapiens SCN2A protein
MAQSVLVPPGPDSFRFFTRESLAAIEQRIAEEKAKRPKQERKDEDDENGPKPNSDLEAGKSLPFIYGDIPPEMVSVPLEDLDPYYINKKTFIVLNKGKAISRFSATPALYILTPFNPIRKLAIKILVHSLFNMLIMCTILTNCVFMTMSNPPDWTKNVEYTFTGIYTFESLIKILARGFCLEDFTFLRDPWNWLDFTVITFAYVTEFVDLGNVSALRTFRVLRALKTISVIPGLKTIVGALIQSVKKLSDVMILTVFCLSVFALIGLQLFMGNLRNKCLQWPPDNSSFEINITSFFNNSLDGNGTTFNRTVSIFNWDEYIEDKSHFYFLEGQNDALLCGNSSDAGQCPEGYICVKAGRNPNYGYTSFDTFSWAFLSLFRLMTQDFWENLYQLTLRAAGKTYMIFFVLVIFLGSFYLINLILAVVAMAYEEQNQATLEEAEQKEAEFQQMLEQLKKQQEEAQAAAAAASAESRDFSGAGGIGVFSESSSVASKLSSKSEKELKNRRKKKKQKEQSGEEEKNDRVRKSESEDSIRRKGFRFSLEGSRLTYEKRFSSPHQSLLSIRGSLFSPRRNSRASLFSFRGRAKDIGSENDFADDEHSTFEDNDSRRDSLFVPHRHGERRHSNVSQASRASRVLPILPMNGKMHSAVDCNGVVSLVGGPSTLTSAGQLLPEGTTTETEIRKRRSSSYHVSMDLLEDPTSRQRAMSIASILTNTMEELEESRQKCPPCWYKFANMCLIWDCCKPWLKVKHLVNLVVMDPFVDLAITICIVLNTLFMAMEHYPMTEQFSSVLSVGNLVFTGIFTAEMFLKIIAMDPYYYFQEGWNIFDGFIVSLSLMELGLANVEGLSVLRSFRLLRVFKLAKSWPTLNMLIKIIGNSVGALGNLTLVLAIIVFIFAVVGMQLFGKSYKECVCKISNDCELPRWHMHDFFHSFLIVFRVLCGEWIETMWDCMEVAGQTMCLTVFMMVMVIGNLVVLNLFLALLLSSFSSDNLAATDDDNEMNNLQIAVGRMQKGIDFVKRKIREFIQKAFVRKQKALDEIKPLEDLNNKKDSCISNHTTIEIGKDLNYLKDGNGTTSGIGSSVEKYVVDESDYMSFINNPSLTVTVPIAVGESDFENLNTEEFSSESDMEESKEKLNATSSSEGSTVDIGAPAEGEQPEVEPEESLEPEACFTEDCVRKFKCCQISIEEGKGKLWWNLRKTCYKIVEHNWFETFIVFMILLSSGALAFEDIYIEQRKTIKTMLEYADKVFTYIFILEMLLKWVAYGFQVYFTNAWCWLDFLIVDVSLVSLTANALGYSELGAIKSLRTLRALRPLRALSRFEGMRVVVNALLGAIPSIMNVLLVCLIFWLIFSIMGVNLFAGKFYHCINYTTGEMFDVSVVNNYSECKALIESNQTARWKNVKVNFDNVGLGYLSLLQVATFKGWMDIMYAAVDSRNVELQPKYEDNLYMYLYFVIFIIFGSFFTLNLFIGVIIDNFNQQKKKFGGQDIFMTEEQKKYYNAMKKLGSKKPQKPIPRPANKFQGMVFDFVTKQVFDISIMILICLNMVTMMVETDDQSQEMTNILYWINLVFIVLFTGECVLKLISLRYYYFTIGWNIFDFVVVILSIVGMFLAELIEKYFVSPTLFRVIRLARIGRILRLIKGAKGIRTLLFALMMSLPALFNIGLLLFLVMFIYAIFGMSNFAYVKREVGIDDMFNFETFGNSMICLFQITTSAGWDGLLAPILNSGPPDCDPDKDHPGSSVKGDCGNPSVGIFFFVSYIIISFLVVVNMYIAVILENFSVATEESAEPLSEDDFEMFYEVWEKFDPDATQFIEFAKLSDFADALDPPLLIAKPNKVQLIAMDLPMVSGDRIHCLDILFAFTKRVLGESGEMDALRIQMEERFMASNPSKVSYEPITTTLKRKQEEVSAIIIQRAYRRYLLKQKVKKVSSIYKKDKGKECDGTPIKEDTLIDKLNENSTPEKTDMTPSTTSPPSYDSVTKPEKEKFEKDKSEKEDKGKDIRESKK

SEQ ID NO: 2 - DNA, 8630 bp, Homo sapiens
  misc_feature 1..8630: note = Homo sapiens SCN2A RNA
  source 1..8630: mol_type = genomic DNA
aagcatgatggaattttagctgcagtcttcttggtgccagcttatcaatcccaaactctgggtgtaaaagattctacagggcactttcttatgcaaggagctaaacagtgattaaaggagcaggatgaaaagatggcacagtcagtgctggtaccgccaggacctgacagcttccgcttctttaccagggaatcccttgctgctattgaacaacgcattgcagaagagaaagctaagagacccaaacaggaacgcaaggatgaggatgatgaaaatggcccaaagccaaacagtgacttggaagcaggaaaatctcttccatttatttatggagacattcctccagagatggtgtcagtgcccctggaggatctggacccctactatatcaataagaaaacgtttatagtattgaataaagggaaagcaatctctcgattcagtgccacccctgccctttacattttaactcccttcaaccctattagaaaattagctattaagattttggtacattctttattcaatatgctcattatgtgcacgattcttaccaactgtgtatttatgaccatgagtaaccctccagactggacaaagaatgtggagtatacctttacaggaatttatacttttgaatcacttattaaaatacttgcaaggggcttttgtttagaagatttcacatttttacgggatccatggaattggttggatttcacagtcattacttttgcatatgtgacagagtttgtggacctgggcaatgtctcagcgttgagaacattcagagttctccgagcattgaaaacaatttcagtcattccaggcctgaagaccattgtgggggccctgatccagtcagtgaagaagctttctgatgtcatgatcttgactgtgttctgtctaagcgtgtttgcgctaataggattgcagttgttcatgggcaacctacgaaataaatgtttgcaatggcctccagataattcttcctttgaaataaatatcacttccttctttaacaattcattggatgggaatggtactactttcaataggacagtgagcatatttaactgggatgaatatattgaggataaaagtcacttttattttttagaggggcaaaatgatgctctgctttgtggcaacagctcagatgcaggccagtgtcctgaaggatacatctgtgtgaaggctggtagaaaccccaactatggctacacgagctttgacacctttagttgggcctttttgtccttatttcgtctcatgactcaagacttctgggaaaacctttatcaactgacactacgtgctgctgggaaaacgtacatgatattttttgtgctggtcattttcttgggctcattctatctaataaatttgatcttggctgtggtggccatggcctatgaggaacagaatcaggccacattggaagaggctgaacagaaggaagctgaatttcagcagatgctcgaacagttgaaaaagcaacaagaagaagctcaggcggcagctgcagccgcatctgctgaatcaagagacttcagtggtgctggtgggataggagttttttcagagagttcttcagtagcatctaagttgagctccaaaagtgaaaaagagctgaaaaacagaagaaagaaaaagaaacagaaagaacagtctggagaagaagagaaaaatgacagagtccgaaaatcggaatctgaagacagcataagaagaaaaggtttccgtttttccttggaaggaagtaggctgacatatgaaaagagattttcttctccacaccagtccttactgagcatccgtggctcccttttctctccaagacgcaacagtagggcgagccttttcagcttcagaggtcgagcaaaggacattggctctgagaatgactttgctgatgatgagcacagcacctttgaggacaatgacagccgaagagactctctgttcgtgccgcacagacatggagaacggcgccacagcaatgtcagccaggccagccgtgcctccagggtgctccccatcctgcccatgaatgggaagatgcatagcgctgtggactgcaatggtgtggtctccctggtcgggggcccttctaccctcacatctgctgggcagctcctaccagagggcacaactactgaaacagaaataagaaagagacggtccagttcttatcatgtttccatggatttattggaagatcctacatcaaggcaaagagcaatgagtatagccagtattttgaccaacaccatggaagaacttgaagaatccagacagaaatgcccaccatgctggtataaatttgctaatatgtgtttgatttgggactgttgtaaaccatggttaaaggtgaaacaccttgtcaacctggttgtaatggacccatttgttgacctggccatcaccatctgcattgtcttaaatacactcttcatggctatggagcactatcccatgacggagcagttcagcagtgtactgtctgttggaaacctggtcttcacagggatcttcacagcagaaatgtttctcaagataattgccatggatccatattattactttcaagaaggctggaatatttttgatggttttattgtgagccttagtttaatggaacttggtttggcaaatgtggaaggattgtcagttctccgatcattccggctgctccgagttttcaagttggcaaaatcttggccaactctaaatatgctaattaagatcattggcaattctgtgggggctctaggaaacctcaccttggtattggccatcatcgtcttcatttttgctgtggtcggcatgcagctctttggtaagagctacaaagaatgtgtctgcaagatttccaatgattgtgaactcccacgctggcacatgcatgactttttccactccttcctgatcgtgttccgcgtgctgtgtggagagtggatagagaccatgtgggactgtatggaggtcgctggccaaaccatgtgccttactgtcttcatgatggtcatggtgattggaaatctagtggttctgaacctcttcttggccttgcttttgagttccttcagttctgacaatcttgctgccactgatgatgataacgaaatgaataatctccagattgctgtgggaaggatgcagaaaggaatcgattttgttaaaagaaaaatacgtgaatttattcagaaagcctttgttaggaagcagaaagctttagatgaaattaaaccgcttgaagatctaaataataaaaaagacagctgtatttccaaccataccaccatagaaataggcaaagacctcaattatctcaaagacggaaatggaactactagtggcataggcagcagtgtagaaaaatatgtcgtggatgaaagtgattacatgtcatttataaacaaccctagcctcactgtgacagtaccaattgctgttggagaatctgactttgaaaatttaaatactgaagaattcagcagcgagtcagatatggaggaaagcaaagagaagctaaatgcaactagttcatctgaaggcagcacggttgatattggagctcccgccgagggagaacagcctgaggttgaacctgaggaatcccttgaacctgaagcctgttttacagaagactgtgtacggaagttcaagtgttgtcagataagcatagaagaaggcaaagggaaactctggtggaatttgaggaaaacatgctataagatagtggagcacaattggttcgaaaccttcattgtcttcatgattctgctgagcagtggggctctggcctttgaagatatatacattgagcagcgaaaaaccattaagaccatgttagaatatgctgacaaggttttcacttacatattcattctggaaatgctgctaaagtgggttgcatatggttttcaagtgtattttaccaatgcctggtgctggctagacttcctgattgttgatgtctcactggttagcttaactgcaaatgccttgggttactcagaacttggtgccatcaaatccctcagaacactaagagctctgaggccactgagagctttgtcccggtttgaaggaatgagggttgttgtaaatgctcttttaggagccattccatctatcatgaatgtacttctggtttgtctgatcttttggctaatattcagtatcatgggagtgaatctctttgctggcaagttttaccattgtattaattacaccactggagagatgtttgatgtaagcgtggtcaacaactacagtgagtgcaaagctctcattgagagcaatcaaactgccaggtggaaaaatgtgaaagtaaactttgataacgtaggacttggatatctgtctctacttcaagtagccacgtttaagggatggatggatattatgtatgcagctgttgattcacgaaatgtagaattacaacccaagtatgaagacaacctgtacatgtatctttattttgtcatctttattatttttggttcattctttaccttgaatcttttcattggtgtcatcatagataacttcaaccaacagaaaaagaagtttggaggtcaagacatttttatgacagaagaacagaagaaatactacaatgcaatgaaaaaactgggttcaaagaaaccacaaaaacccatacctcgacctgctaacaaattccaaggaatggtctttgattttgtaaccaaacaagtctttgatatcagcatcatgatcctcatctgccttaacatggtcaccatgatggtggaaaccgatgaccagagtcaagaaatgacaaacattctgtactggattaatctggtgtttattgttctgttcactggagaatgtgtgctgaaactgatctctcttcgttactactatttcactattggatggaatatttttgattttgtggtggtcattctctccattgtaggaatgtttctggctgaactgatagaaaagtattttgtgtcccctaccctgttccgagtgatccgtcttgccaggattggccgaatcctacgtctgatcaaaggagcaaaggggatccgcacgctgctctttgctttgatgatgtcccttcctgcgttgtttaacatcggcctccttcttttcctggtcatgttcatctacgccatctttgggatgtccaattttgcctatgttaagagggaagttgggatcgatgacatgttcaactttgagacctttggcaacagcatgatctgcctgttccaaattacaacctctgctggctgggatggattgctagcacctattcttaatagtggacctccagactgtgaccctgacaaagatcaccctggaagctcagttaaaggagactgtgggaacccatctgttgggattttcttttttgtcagttacatcatcatatccttcctggttgtggtgaacatgtacatcgcggtcatcctggagaacttcagtgttgctactgaagaaagtgcagagcctctgagtgaggatgactttgagatgttctatgaggtttgggagaagtttgatcccgatgcgacccagtttatagagtttgccaaactttctgattttgcagatgccctggatcctcctcttctcatagcaaaacccaacaaagtccagctcattgccatggatctgcccatggtgagtggtgaccggatccactgtcttgacatcttatttgcttttacaaagcgtgttttgggtgagagtggagagatggatgcccttcgaatacagatggaagagcgattcatggcatcaaacccctccaaagtctcttatgagcccattacgaccacgttgaaacgcaaacaagaggaggtgtctgctattattatccagagggcttacagacgctacctcttgaagcaaaaagttaaaaaggtatcaagtatatacaagaaagacaaaggcaaagaatgtgatggaacacccatcaaagaagatactctcattgataaactgaatgagaattcaactccagagaaaaccgatatgacgccttccaccacgtctccaccctcgtatgatagtgtgaccaaaccagaaaaagaaaaatttgaaaaagacaaatcagaaaaggaagacaaagggaaagatatcagggaaagtaaaaagtaaaaagaaaccaagaattttccattttgtgatcaattgtttacagcccgtgatggtgatgtgtttgtgtcaacaggactcccacaggaggtctatgccaaactgactgtttttacaaatgtatacttaaggtcagtgcctataacaagacagagacctctggtcagcaaactggaactcagtaaactggagaaatagtatcgatgggaggtttctattttcacaaccagctgacactgctgaagagcagaggcgtaatggctactcagacgataggaaccaatttaaaggggggagggaagttaaatttttatgtaaattcaacatgtgacacttgataatagtaattgtcaccagtgtttatgttttaactgccacacctgccatatttttacaaaacgtgtgctgtgaatttatcacttttctttttaattcacaggttgtttactattatatgtgactatttttgtaaatgggtttgtgtttggggagagggattaaagggagggaattctacatttctctattgtattgtataactggatatattttaaatggaggcatgctgcaattctcattcacacataaaaaaatcacatcacaaaagggaagagtttacttcttgtttcaggatgtttttagatttttgaggtgcttaaatagctattcgtatttttaaggtgtctcatccagaaaaaatttaatgtgcctgtaaatgttccatagaatcacaagcattaaagagttgttttatttttacataacccattaaatgtacatgtatatatgtatatatgtatatgtgcgtgtatatacatatatatgtatacacacatgcacacacagagatatacacataccattacattgtcattcacagtcccagcagcatgactatcacatttttgataagtgtcctttggcataaaataaaaatatcctatcagtcctttctaagaagcctgaattgaccaaaaaacatccccaccaccactttataaagttgattctgctttatcctgcagtattgtttagccatcttctgctcttggtaaggttgacatagtatatgtcaatttaaaaaataaaagtctgctttgtaaatagtaattttacccagtggtgcatgtttgagcaaacaaaaatgatgatttaagcacactacttattgcatcaaatatgtaccacagtaagtatagtttgcaagctttcaacaggtaatatgatgtaattggttccattatagtttgaagctgtcactgctgcatgtttatcttgcctatgctgctgtatcttattccttccactgttcagaagtctaatatgggaagccatatatcagtggtaaagtgaagcaaattgttctaccaagacctcattcttcatgtcattaagcaataggttgcagcaaacaaggaagagcttcttgctttttattcttccaaccttaattgaacactcaatgatgaaaagcccgactgtacaaacatgttgcaagctgcttaaatctgtttaaaatatatggttagagttttctaagaaaatataaatactgtaaaaagttcattttattttatttttcagccttttgtacgtaaaatgagaaattaaaagtatcttcaggtggatgtcacagtcactattgttagtttctgttcctagcacttttaaattgaagcacttcacaaaataagaagcaaggactaggatgcagtgtaggtttctgcttttttattagtactgtaaacttgcacacatttcaatgtgaaacaaatctcaaactgagttcaatgtttatttgctttcaatagtaatgccttatcattgaaagaggcttaaagaaaaaaaaaatcagctgatactcttggcattgcttgaatccaatgtttccacctagtctttttattcagtaatcatcagtcttttccaatgtttgtttacacagatagatcttattgacccatatggcactagaactgtatcagatataatatgggatcccagctttttttcctctcccacaaaaccaggtagtgaagttatattaccagttacagcaaaatactttgtgtttcacaagcaacaataaatgtagattctttatactgaagctattgacttgtagtgtgttggtgaaatgcatgcaggaaaatgctgttaccataaagaacggtaaaccacattacaatcaagccaaaagaataaaggtttcgcttttgtttttgtatttaattgttgtctttgtttctatctttgaaatgccatttaaaggtagatttctatcatgtaaaaataatctatctgaaaaacaaatgtaaagaacacacattaattactataattcatctttcaattttttcatggaatggaagttaattaagaagagtgtattggataactactttaatattggccaaaaagctagatatggcatcaggtagactagtggaaagttacaaaaattaataaaaaattgactaaca

(19) United States
(12) Patent Application Publication
(10) Pub. No.: US 2025/0146073 A1
(43) Pub. Date: May 8, 2025

(54) METHODS FOR THE TREATMENT OF SCN2A-RELATED DISORDERS

(71) Applicant: Praxis Precision Medicines, Inc., Boston, MA (US)

(72) Inventor: Kelley Dalby, Cambridge, MA (US)

(21) Appl. No.: 18/834,440

(22) PCT Filed: Feb. 3, 2023
(86) PCT No.: PCT/US2023/061988

§ 371 Date: Jul. 30, 2024

Related U.S. Application Data
(60) Provisional application No. 63/349,318, filed on Jun. 6, 2022.
(60) Provisional application No. 63/306,834, filed on Feb. 4, 2022.

(51) Int. Cl. C12Q 1/6883

(52) U.S. Cl. CPC C12Q 1/6883; C12Q 2600/112

(57) ABSTRACT

The present disclosure features useful methods to treat a SCN2A-related disorder caused by a gain-of-function SCN2A mutation using an SCN2A inhibitor. The present disclosure also features useful methods to treat a SCN2A-related disorder caused by a loss-of-function SCN2A mutation using an SCN2A enhancer. By monitoring the early onset seizure status of a subject, appropriate treatments can be administered more quickly without having to verify whether the subject has a gain-of-function or loss-of-function SCN2A mutation.

### FIELD

The present disclosure is directed, in certain aspects, to compositions and methods for treating SCN2A-related disorders in a subject in need thereof, including methods of treating a SCN2A-related disorder associated with a gain-of-function SCN2A mutation using an SCN2A inhibitor and methods of treating a SCN2A-related disorder associated with a loss-of-function SCN2A mutation using an SCN2A enhancer.

### BACKGROUND

The current standard of care for treatment of patients with pediatric neurological disorders includes genetic testing to determine if the patient has an SCN2A mutation. If the patient has an SCN2A mutation, the clinician may search the literature to assess what is known about the particular SCN2A mutation, including whether it is a gain-of-function or loss-of-function mutation or conduct biochemical testing to determine whether the SCN2A mutation is a gain-of-function or loss-of-function mutation. Before it is known whether the SCN2A mutation is a gain-of-function or loss-of function mutation, a trial-and-error procedure may be used to prescribe different medications until a medication is identified that treats the patient's symptoms with manageable side effects. Alternatively, treatment may be delayed until the status of the SCN2A mutation has been confirmed as either a gain-of-function or loss-of function mutation by genetic or biochemical testing.

Currently, compositions for treating SCN2A-related disorders are being developed that inhibit or enhance SCN2A activity. Ideally, these SCN2A inhibitors and SCN2A enhancers will reduce or even eliminate the need for trial-and-error experimentation to find an effective medication and provide specific treatments that target the actual cause of the SCN2A-related disorder and produce fewer side effects. It may be desirable when using such SCN2A inhibitors to ensure that the subject being treated has a SCN2A gain-of-function mutation, and when using such SCN2A enhancers to ensure that the subject being treated has a SCN2A loss-of-function mutation. Therefore, there is a need for improved methods of treating SCN2A-related disorders that do not require experimental verification of whether the SCN2A mutation the subject carries is a gain-of-function mutation or a loss-of-function mutation before starting treatment with an SCN2A inhibitor or an SCN2A enhancer. By eliminating the need to verify the status of the SCN2A mutation, patients with SCN2A-related disorders, and particularly pediatric patients, can receive effective treatments sooner.

### SUMMARY

A first aspect of the present disclosure includes methods for treating a subject with a SCN2A-related disorder caused by a gain-of-function SCN2A mutation, comprising determining whether the subject has had an early onset seizure; administering a SCN2A inhibitor to the subject if the subject has had an early onset seizure, wherein the method does not comprise experimentally verifying that the subject's SCN2A mutation is a gain-of-function mutation or confirming that the subject's SCN2A mutation has previously been experimentally verified as a gain-of-function mutation prior to administering the SCN2A inhibitor. In some embodiments, the early onset seizure occurred within the first three months, the first two and a half months, the first two months, the first month and a half, the first month, the first four weeks, the first three weeks, the first two weeks, the first week, or the first day of the subject's life. In certain embodiments, the early onset seizure occurred before birth, while the subject was in utero. Certain embodiments, which may be combined with any or all of the above embodiments, include determining that the subject carries the SCN2A mutation prior to administering the SCN2A inhibitor or after administering the SCN2A inhibitor. In some embodiments that may be combined with any or all of the above embodiments, the SCN2A mutation is A263V, E430A, R1882Q, G879R, G1593R, K1502N, V1601L, G211D, S1780I, D343H, or combinations of the foregoing. In some embodiments that may be combined with any or all of the above embodiments, the subject is less than 18 years old. In some embodiments that may be combined with any or all of the above embodiments, the SCN2A-related disorder is Ohtahara syndrome, epilepsy of infancy with migrating focal seizures, or early onset epileptic encephalopathy. In some embodiments that may be combined with any or all of the above embodiments, the SCN2A inhibitor is administered intrathecally, intramedullarly, intracerebroventricularly, or parenterally. In some embodiments of the preceding embodiments, the parenteral administration is subcutaneous, intravenous, or intramuscular injection or infusion. In some embodiments that may be combined with any or all of the above embodiments, the SCN2A inhibitor is a CRISPR-Cas repressor, a sodium channel blocker (e.g., a Naᵥ1.2 sodium channel blocker), an antibody, or a nucleic acid inhibitor molecule that targets the SCN2A gene, such as an antisense oligonucleotide or an siRNA. In some embodiments of the preceding embodiments, the method decreases expression of the human SCN2A gene. In some embodiments, the method comprises inhibiting the expression of SCN2A in neuronal cells in the subject.

A second aspect of the present disclosure includes compositions comprising an SCN2A inhibitor for use in the above method aspect and any and all of the embodiments thereof. In certain embodiments, the SCN2A inhibitor comprises a CRISPR-Cas repressor, a sodium channel blocker (e.g., a Naᵥ1.2 sodium channel blocker), an antibody, or a nucleic acid inhibitor molecule that targets the SCN2A gene, such as an antisense oligonucleotide or an siRNA.

A third aspect of the present disclosure includes uses of an SCN2A inhibitor for treating a subject with a SCN2A-related disorder caused by a gain-of-function SCN2A mutation according to the above method aspect and any and all of the embodiments thereof. In certain embodiments, the SCN2A inhibitor comprises a CRISPR-Cas repressor, a sodium channel blocker (e.g., a Naᵥ1.2 sodium channel blocker), an antibody, or a nucleic acid inhibitor molecule that targets the SCN2A gene, such as an antisense oligonucleotide or an siRNA.

A fourth aspect of the disclosure includes methods for treating a subject with a SCN2A-related disorder caused by a loss-of-function SCN2A mutation, comprising determining whether the subject has had an early onset seizure; administering a SCN2A enhancer to the subject if the subject has not had an early onset seizure, wherein the SCN2A enhancer is administered as a second line therapy or is co-administered with a first line therapy if the subject has infantile spasms, and wherein the method does not comprise experimentally verifying the subject's SCN2A mutation as a loss-of-function mutation or confirming that the subject's SCN2A mutation has previously been experimentally verified as a loss-of-function mutation prior to administering the SCN2A enhancer. In some embodiments, the first line therapy is selected from steroids, adrenocorticotropic hormone (ACTH), and vigabatrin. In some embodiments that may be combined with the above embodiments, the subject had no seizure within the first six months, within the first five months, within the first four months, within the first three months, the first two and a half months, or the first two months of the subject's life. Certain embodiments, which may be combined with any or all of the above embodiments, include determining that the subject carries the SCN2A mutation prior to administering the SCN2A enhancer or after administering the SCN2A enhancer. In some embodiments that may be combined with any or all of the above embodiments, the SCN2A mutation comprises M951R, F1375V, R853Q, A1773T, R571H, K1422E, D195G, Y428, K507E, Y1771H, S1758R, or combinations of the foregoing. In some embodiments that may be combined with any or all of the above embodiments, the subject is less than 18 years old. In some embodiments that may be combined with any or all of the above embodiments, the SCN2A-related disorder is autism spectrum disorder, benign familial neonatal/infantile seizures, infantile spasms, Ohtahara syndrome, late seizure onset epileptic encephalopathy, or epilepsy of infancy with migrating focal seizures. In some embodiments that may be combined with any or all of the above embodiments, the SCN2A enhancer is administered intrathecally, intramedullarly, intracerebroventricularly, or parenterally. In some embodiments of the preceding embodiment, the parenteral administration is subcutaneous, intravenous, or intramuscular injection or infusion. In some embodiments that may be combined with any or all of the above embodiments, the SCN2A enhancer is a nucleic acid inhibitor molecule that targets the SCN2A gene, such as an antisense oligonucleotide, a CRISPR-Cas enhancer, or a SCN2A activator, such a small molecule SCN2A activator. In certain embodiments, the method increases expression of the human SCN2A gene. In some embodiments, the method comprises increasing the expression of SCN2A in neuronal cells in the subject.

A fifth aspect of the present disclosure includes compositions comprising an SCN2A enhancer for use in the above method aspect and any and all of the embodiments thereof. In certain embodiments, the SCN2A enhancer comprises a nucleic acid inhibitor molecule that targets the SCN2A gene, such as an antisense oligonucleotide or an siRNA, CRISPR-Cas enhancer, or a SCN2A activator, such a small molecule SCN2A activator.

A sixth aspect of the present disclosure includes uses of an SCN2A enhancer for treating a subject with a SCN2A-related disorder caused by a loss-of-function SCN2A mutation according to the the above method aspect and any and all of the embodiments thereof. In certain embodiments, the SCN2A enhancer comprises a nucleic acid inhibitor molecule that targets the SCN2A gene, such as an antisense oligonucleotide or an siRNA, CRISPR-Cas enhancer, or a SCN2A activator, such a small molecule SCN2A activator.

In some embodiments of all aspects disclosed herein, the antisense oligonucleotide comprises a single-stranded oligonucleotide that is 10-80 nucleosides in length and having a nucleobase sequence comprising a portion of 10 contiguous nucleobases having at least 80% complementary to an equal length portion of a target region of a pre-mRNA transcript or an mRNA transcript of a human SCN2A gene, in an amount and for a duration sufficient to treat the SCN2A-related disorder. In some embodiments of all aspects disclosed herein, the oligonucleotide comprises, consists essentially of, or consists of a nucleobase sequence complementary to a portion of SCN2A mRNA that encodes the amino acid sequence of GenBank accession no. NP_066287.2 (SEQ ID NO: 1) or comprises the nucleobase sequence of GenBank accession no. NM_021007.3 (SEQ ID NO: 2). In some embodiments of all aspects disclosed herein, the oligonucleotide comprises one or more modified sugar, one or more modified internucleoside linkages, and/or one or more modified nucleobases. In some embodiments of all aspects disclosed herein, the oligonucleotide comprises one or more modified sugars, which may be independently selected from the group consisting of a bicyclic sugar, a 2′-O-methoxyethyl (2MOE) modified sugar, a 2′-O-methyl (2-OMe) modified sugar, a 2′-methoxy modified sugar, a 2′-Fluoro modified sugar, a 2′-O-alkyl modified sugar, a constrained ethyl (cEt) modified sugar, a locked sugar, and an unlocked sugar. In some embodiments of the preceding embodiment, each nucleoside of the oligonucleotide comprises a 2MOE modified sugar. In some embodiments of all aspects disclosed herein, the oligonucleotide comprises one or more modified internucleoside linkages, which may comprise a modified phosphate, such as a phosphorothioate, a phosphorodithioate, a phosphoramidate, a phosphorodiamidate, a thiophosphoramidate, a thiophosphorodiamidate, a methyl phosphonate, a phosphoromorpholidate, and a phosphoropiperazidate. In some embodiments of all aspects disclosed herein, the oligonucleotide has phosphorothioate internucleoside linkages throughout the length of the oligonucleotide. In some embodiments of all aspects disclosed herein, the oligonucleotide has phosphorodiamidate morpholino internucleoside linkages throughout the length of the oligonucleotide. In some embodiments of all aspects disclosed herein, the oligonucleotide comprises one or more modified nucleobases, such as, 5-methylcytosine, 5-hydroxymethyl cytosine, xanthine, hypoxanthine, 2-aminoadenine, 6-methyladenine, 6-methylguanine, 2-propyladenine, 2-propylguanine, 2-thiouracil, 2-thiothymine, 2-thiocytosine, 5-halouracil, 5-halocytosine, 5-propynyluracil, 5-propynylcytosine, 6-azouracil, 6-azocytosine, 6-azothymine, 5-uracil (pseudouracil), 4-thiouracil, 8-haloadenine, 8-aminoadenine, 8-thioladenine, 8-thioalkyladenine, 8-hydroxyladenine, 8-haloguanine, 8-aminoguanine, 8-thiolguanine, 8-thioalkylguanine, 8-hydroxylguanine, 5-bromouracil, 5-trifluoromethyluracil, 5-bromocytosine, 5-trifluoromethylcytosine, 7-methylguanine, 7-methyladenine, 2-fluoroadenine, 8-azaguanine, 8-azaadenine, 7-deazaguanine, 7-deazaadenine, 3-deazaguanine, and 3-deazaadenine. In some embodiments of the preceding embodiment, the modified nucleobase is a 5-methylcytosine. In some embodiments of the preceding embodiment, each cytosine of the oligonucleotide is a 5-methylcytosine. In some embodiments of all aspects disclosed herein, the modified oligonucleotide comprises: a gap segment consisting of linked deoxynucleosides; a 5′ wing segment consisting of linked nucleosides; and a 3′ wing segment consisting of linked nucleosides; wherein the gap segment is positioned immediately adjacent to and between the 5′ wing segment and the 3′ wing segment and wherein each nucleoside of each wing segment comprises a modified sugar. In some embodiments of all aspect disclosed herein, the oligonucleotide consists of 12 to 40 nucleobases. In some embodiments of the preceding embodiments, the oligonucleotide consists of 16 to 30 nucleobases. In some embodiments of all aspects disclosed herein, the oligonucleotide is selective for SCN2A pre-mRNA or mRNA over SCN1A pre-mRNA or mRNA.

### BRIEF DESCRIPTION OF THE DRAWINGS

FIG. 1 shows a flowchart of the overall experimental design as described in Example 1.

FIGS. 2A-D show characteristics of patients selected for the analysis, as described in Example 1. FIG. 2A is a pie chart showing the percentage of patients in the cohort by phenotype. FIG. 2B is a bar graph showing the patient sex by phenotype. FIG. 2C is a bar graph showing the median age of seizure onset in days for the three phenotypic classes characterized by having seizures. FIG. 2D is a bar graph showing the age at the time of data collection in years by phenotypic class with the average age at the right for each phenotypic class. FIG. 2E is a plot showing the seizure history from birth to 4.5 years of age of patients in the cohort. The age (in days) of seizure onset is shown on the left most column of numbers. The age in years at the time of data collection is shown in the column to the right. The dashed line from top to bottom shows the cut-off of three months from birth. FIG. 2F shows the current (as of the time of data collection) and prior medication counts for each patient, indicating that the patients across all phenotypic classes with seizures received a number of medications, including both general sodium channel blocker antiepileptic drugs (Sodium Ch. AED), other antiepileptic drugs (Other AED), and non-antiepileptic drugs (Non-AED). In contrast, autism spectrum disorder without seizures were largely treated with non-antiepileptic drugs. The phenotypic classes are grouped as (i) early onset seizures (EO), (ii) late onset seizures without infantile spasms (LO), (ii) late onset seizures with infantile spasms (LOIS), and (iv) autism spectrum disorder without seizures (AO). The age in years at the time of data collection is shown in the column on the left.

FIG. 3 shows the experimental verification based on dynamic clamp (DC) analysis as discussed in Example 1 of SCN2A mutations for gain-of-function (GoF) or loss-of-function (LoF) and the corresponding phenotype of (i) early onset seizures (EO), (ii) late onset seizures with infantile spasms (LO (w/ IS)), (iii) late onset without infantile spasms (LO), and (iv) autism spectrum disorder without seizures (ASD (no epilepsy)).

### DETAILED DESCRIPTION

Categorizing Patients as Having Gain-of-Function or Loss-of-Function SCN2A Mutations

The present disclosure is based, in part, upon the finding that all analyzed patients with an experimentally verified gain-of-function SCN2A mutation experienced their first seizure shortly after birth. This finding contributed to the improved methods of treatment disclosed herein that do not require experimental verification that the subject carries a gain-of-function SCN2A mutation before treating the patient with a SCN2A inhibitor. A subject with any SCN2A mutation that experiences a seizure within the first few months or even weeks of life may immediately begin treatment with a SCN2A inhibitor without requiring further experimental verification of the SCN2A mutation as a gain-of-function mutation. Likewise, a subject with a SCN2A mutation that experiences a seizure in utero may begin treatment with a SCN2A inhibitor without requiring further experimental verification of the SCN2A mutation as a gain-of-function mutation.

This application discloses methods of using a SCN2A inhibitor to treat SCN2A-related disorders in a subject having elevated SCN2A levels or elevated SCNA2 activity, where the methods do not require experimental verification of the subject's SCN2A mutation as a gain-of-function mutation or confirmation that the subject's SCN2A mutation has previously been experimentally verified as a gain-of-function mutation prior to administering the SCN2A inhibitor.

Given the identified link between early onset seizures and a gain-of-function SCN2A mutation, a subject who has a SCN2A mutation but does not experience an early onset seizure is more likely to have a loss-of-function SCN2A mutation. Indeed, all analyzed patients who had their first seizure after their first three months of life or who did not experience a seizure were identified as having loss-of-function SCN2A mutations. As such, a SCN2A enhancer may be used in a subject having reduced SCN2A levels or reduced SCNA2 activity, where the methods do not require experimental verification of the subject's SCN2A mutation as a loss-of-function mutation or confirmation that the subject's SCN2A mutation has previously been experimentally verified as a loss-of-function mutation prior to administering the SCN2A enhancer. In such methods, a subject with any SCN2A mutation that does not experience a seizure within the first few months, or even weeks, of life may immediately begin treatment with an SCN2A enhancer without requiring experimental verification of the SCN2A mutation as a loss-of-function mutation before treating with the SCN2A enhancer.

This application also discloses methods of using a SCN2A enhancer to treat SCN2A-related disorders in a subject having reduced SCN2A levels or reduced SCN2A activity, where the methods do not require experimental verification of the subject's SCN2A mutation as a loss-of-function mutation or confirming that the subject's SCN2A mutation has previously been experimentally verified as a loss-of-function mutation prior to administering the SCN2A enhancer.

The human SCN2A gene (UniProt: Q99250, NP_066287.2) (SEQ ID NO: 1) encodes Naᵥ1.2, the alpha subunit of a voltage-gated sodium channel that is expressed in the brain and involved in neural signaling. Mutations in SCN2A may be gain-of-function mutations or loss-of-function mutations that are associated with benign familial neonatal/infantile seizures (BFNIS), autism, including autism with and without epilepsy, schizophrenia, intellectual disability, infantile spasms, and epileptic encephalopathies, including Ohtahara syndrome, epilepsy of infancy with migrating focal seizures (EIMFS), late seizure onset epileptic encephalopathy (e.g., LOE), and early onset epileptic encephalopathy (EOEE). See Howell, Katherine B., et al. “SCN2A encephalopathy: A major cause of epilepsy of infancy with migrating focal seizures.” Neurology 85.11 (2015): 958-966; Dhamija, Radhika, et al. “Novel de novo SCN2A mutation in a child with migrating focal seizures of infancy.” Pediatric neurology 49.6 (2013): 486-488; Nakamura, Kazuyuki, et al. “Clinical spectrum of SCN2A mutations expanding to Ohtahara syndrome.” Neurology 81.11 (2013): 992-998; Baasch, Ann-Lena, et al. “Exome sequencing identifies a de novo SCN2A mutation in a patient with intractable seizures, severe intellectual disability, optic atrophy, muscular hypotonia, and brain abnormalities.” Epilepsia 55.4 (2014): e25-e29.

The methods of treating a subject with a SCN2A-related disorder caused by a gain-of-function SCN2A mutation comprise determining that the subject has had an early onset seizure. If the subject has had an early onset seizure, an SCN2A inhibitor can be administered to that patient. The methods disclosed herein do not require experimentally verifying that the subject's SCN2A mutation as a gain-of-function mutation or confirming that the subject's SCN2A mutation has previously been experimentally verified as a gain-of-function mutation prior to administering the SCN2A inhibitor. In certain embodiments, the early onset seizure occurred within the first three months, the first two and a half months, the first two months, the first month and a half, the first month, the first four weeks, the first three weeks, the first two weeks, the first week, or the first day of the subject's life. In certain embodiments, the early onset seizure occurred before birth, while the subject was in utero. These methods do not exclude administration of SCN2A inhibitors to outlier subjects with a gain-of-function SCN2A mutation who happen not to have had an early onset seizure.

The methods of treating a subject with a SCN2A-related disorder caused by a loss-of-function SCN2A mutation comprise determining that the subject has not had an early onset seizure. If the subject has not had an early onset seizure, an SCN2A enhancer can be administered to that patient. The methods do not require experimentally verifying that the subject's SCN2A mutation as a loss-of-function mutation or confirming that the subject's SCN2A mutation has previously been experimentally verified as a loss-of-function mutation prior to administering the SCN2A enhancer. In certain embodiments, the subject had no seizure within the first six months, within the first five months, within the first four months, within the first three months, the first two and a half months, the first two months, the first month, or the first four weeks of the subject's life.

The loss-of-function SCN2A mutations may be further subdivided into subjects with autism spectrum disorder without seizure, with late onset seizures without infantile spasms, and with late onset seizures with infantile spasms. Subjects with autism spectrum disorder without seizure and with late onset seizures without infantile spasms may immediately begin treatment with an SCN2A enhancer. Subjects with late onset seizures with infantile spasms may in certain embodiments begin a first-line therapy treatment for the infantile spasms owing to the seriousness of infantile spasms. Therefore, the SCN2A enhancer may be administered as a second-line therapy or, where warranted, co-administered with the first-line therapy. In certain embodiments, the first-line therapy is selected from steroids, adrenocorticotropic hormone (ACTH), and vigabatrin. These methods do not exclude administration of SCN2A enhancers to outlier subjects with a loss-of-function SCN2A mutation who happen to have had an early onset seizure.

### Definitions

For convenience, the meaning of some terms and phrases used in the specification, examples, and appended claims are provided below. Unless stated otherwise, or implicit from context, the following terms and phrases include the meanings provided below. The definitions are provided to aid in describing particular embodiments and are not intended to limit the claimed technology. Unless otherwise defined, all technical and scientific terms used herein have the same meaning as commonly understood by one of ordinary skill in the art to which this technology belongs. If there is an apparent discrepancy between the usage of a term in the art and its definition provided herein, the definition provided within the specification shall prevail.

In this application, unless otherwise clear from context, (i) the term “a” may be understood to mean “at least one”; (ii) the term “or” may be understood to mean “and/or”; and (iii) the terms “including” and “comprising” may be understood to encompass itemized components or steps whether presented by themselves or together with one or more additional components or steps.

As used herein, the terms “about” and “approximately” refer to a value that is within 10% above or below the value being described. For example, the term “about 5 nM” indicates a range of from 4.5 to 5.5 nM.

The term “at least” prior to a number or series of numbers is understood to include the number adjacent to the term “at least”, and all subsequent numbers or integers that could logically be included, as clear from context. For example, the number of nucleotides in a nucleic acid molecule must be an integer. For example, “at least 18 nucleotides of a 21-nucleotide nucleic acid molecule” means that 18, 19, 20, or 21 nucleotides have the indicated property. When “at least” is present before a series of numbers or a range, it is understood that “at least” can modify each of the numbers in the series or range.

As used herein, “no more than” or “less than” is understood as the value adjacent to the phrase and logical lower values or integers, as logical from context, to zero. For example, an oligonucleotide with “no more than 3 mismatches to a target sequence” has 3, 2, 1, or 0 mismatches to a target sequence, and an oligonucleotide with “less than 3 mismatches to a target sequence has 2, 1, or 0 mismatches to a target sequence. When “no more than” or “less than” is present before a series of numbers or a range, it is understood that “no more than” or “less than” can modify each of the numbers in the series or range.

As used herein, the term “administration” refers to the administration of a composition (e.g., a compound or a preparation that includes a compound as described herein) to a subject or system. Administration to an animal subject (e.g., to a human) may be by any appropriate route, such as ones described herein. In particular embodiments, administration to a subject involves intrathecal administration of a composition or SCN2A inhibitor or enhancer to the subject.

As used herein, the term “SCN2A” refers to Naᵥ1.2, the alpha subunit of a voltage-gated sodium channel that is expressed in the brain, having an amino acid sequence from any vertebrate or mammalian source, including, but not limited to, human, bovine, chicken, rodent, mouse, rat, porcine, ovine, primate, monkey, and guinea pig, unless specified otherwise. SCN2A can, in some embodiments, include one or more mutations that lead to neurological diseases such as epileptic encephalitis. SCN2A is encoded by the SCN2A gene. In certain embodiments, SCN2A is a human SCN2A represented by SEQ ID NO: 1 as described herein.

As used herein, “target sequence” refers to a contiguous portion of the nucleotide sequence of an mRNA molecule formed during the transcription of a gene, such as an SCN2A gene, including mRNA that is a product of RNA processing of a primary transcription product. In one embodiment, the target portion of the sequence will be at least long enough to serve as a substrate for oligonucleotide-directed (e.g., antisense oligonucleotide (ASO)-directed) cleavage at or near that portion of the nucleotide sequence of an mRNA molecule formed during the transcription of the gene, such as a SCN2A gene. The target sequence may be, for example, from about 9-36 nucleotides in length, such as about 15-30 nucleotides in length, or about 18-22 nucleotides in length. For example, the target sequence can be from about 15-30 nucleotides, such as any of 15-29, 15-28, 15-27, 15-26, 15-25, 15-24, 15-23, 15-22, 15-21, 15-20, 15-19, 15-18, 15-17, 18-30, 18-29, 18-28, 18-27, 18-26, 18-25, 18-24, 18-23, 18-22, 18-21, 18-20, 19-30, 19-29, 19-28, 19-27, 19-26, 19-25, 19-24, 19-23, 19-22, 19-21, 19-20, 20-30, 20-29, 20-28, 20-27, 20-26, 20-25, 20-24, 20-23, 20-22, 20-21, 21-30, 21-29, 21-28, 21-27, 21-26, 21-25, 21-24, 21-23, or 21-22 nucleotides in length. Ranges and lengths intermediate to the above recited ranges and lengths are also contemplated to be part of the embodiments disclosed herein.

As used herein, the term “inhibitor” refers to any agent that reduces the level and/or activity of a protein (e.g., SCN2A or mutated SCN2A). Non-limiting examples of inhibitors include polynucleotides (e.g., oligonucleotides such as ASOs). The term “inhibiting,” as used herein, is used interchangeably with “reducing,” “silencing,” “downregulating,” “suppressing,” and other similar terms, and includes any level of inhibition.

As used herein, the term “enhancer” refers to any agent that increases the level and/or activity of a protein (e.g., SCN2A or mutated SCN2A). Non-limiting examples of enhancers include polynucleotides (e.g., oligonucleotides such as ASOs). The term “enhancing,” as used herein, is used interchangeably with “increasing,” “upregulating,” and other similar terms, and includes any level of enhancement.

The terms “oligonucleotide” and “polynucleotide” as used herein are defined as it is generally understood by the skilled person as a molecule comprising two or more covalently linked nucleosides. Such covalently bound nucleosides may also be referred to as nucleic acid molecules or oligomers. Oligonucleotides are commonly made in the laboratory by solid-phase chemical synthesis followed by purification. When referring to a sequence of the oligonucleotide, reference is made to the sequence or order of nucleobase moieties, or modifications thereof, of the covalently linked nucleotides or nucleosides. The oligonucleotide disclosed herein may be man-made, is chemically synthesized, and is typically purified or isolated. Oligonucleotide is also intended to include (i) compounds that have one or more furanose moieties that are replaced by furanose derivatives or by any structure, cyclic or acyclic, that may be used as a point of covalent attachment for the base moiety, (ii) compounds that have one or more phosphodiester linkages that are either modified, as in the case of phosphoramidate or phosphorothioate linkages, or completely replaced by a suitable linking moiety as in the case of formacetal or riboacetal linkages, and/or (iii) compounds that have one or more linked furanose-phosphodiester linkage moieties replaced by any structure, cyclic or acyclic, that may be used as a point of covalent attachment for the base moiety. The oligonucleotide disclosed herein may comprise one or more alternative nucleosides or nucleotides (e.g., including those described herein). It is also understood that oligonucleotide includes compositions lacking a sugar moiety or nucleobase but still capable of forming a pairing with or hybridizing to a target sequence.

As used herein, the term “nucleic acid inhibitor molecule” refers to an oligonucleotide that reduces or eliminates the expression of a target gene wherein the oligonucleotide contains a region that specifically targets a sequence in the target gene mRNA. Typically, the targeting region of the nucleic acid inhibitor molecule comprises a sequence that is sufficiently complementary to a sequence on the target gene mRNA to direct the effect of the nucleic acid inhibitor molecule to the specified target gene. The nucleic acid inhibitor molecule may include ribonucleotides, deoxyribonucleotides, and/or modified nucleotides.

As used herein, and unless otherwise indicated, the term “complementary,” when used to describe a first nucleotide or nucleoside sequence in relation to a second nucleotide or nucleoside sequence, refers to the ability of an oligonucleotide or polynucleotide comprising the first nucleotide or nucleoside sequence to hybridize and form a duplex structure under certain conditions with an oligonucleotide or polynucleotide comprising the second nucleotide sequence, as will be understood by the skilled person. Such conditions can, for example, be stringent conditions, where stringent conditions can include: 400 mM NaCl, 40 mM PIPES pH 6.4, 1 mM EDTA, 50° C., or 70° C., for 12-16 hours followed by washing (see, e.g., “Molecular Cloning: A Laboratory Manual, Sambrook, et al. (1989) Cold Spring Harbor Laboratory Press). Other conditions, such as physiologically relevant conditions as can be encountered inside an organism, can apply. The skilled person will be able to determine the set of conditions most appropriate for a test of complementarity of two sequences in accordance with the ultimate application of the hybridized nucleotides or nucleosides.

“Complementary” sequences, as used herein, can also include, or be formed entirely from, non-Watson-Crick base pairs and/or base pairs formed from non-natural and alternative nucleotides or nucleosides, in so far as the above requirements with respect to their ability to hybridize are fulfilled. Such non-Watson-Crick base pairs include, but are not limited to, G:U Wobble or Hoogstein base pairing. Complementary sequences between an oligonucleotide and a target sequence as described herein, include base-pairing of the oligonucleotide or polynucleotide comprising a first nucleotide or nucleoside sequence to an oligonucleotide or polynucleotide comprising a second nucleotide or nucleoside sequence over the entire length of one or both nucleotide or nucleoside sequences. Such sequences can be referred to as “fully complementary” with respect to each other herein. However, where a first sequence is referred to as “substantially complementary” with respect to a second sequence herein, the two sequences can be fully complementary, or they can form one or more, but generally not more than 5, 4, 3 or 2, mismatched base pairs upon hybridization for a duplex up to 30 base pairs, while retaining the ability to hybridize under the conditions most relevant to their ultimate application, e.g., inhibition of gene expression via an RNase H-mediated pathway. “Substantially complementary” can also refer to a polynucleotide that is substantially complementary to a contiguous portion of the mRNA of interest (e.g., an mRNA encoding SCN2A). For example, a polynucleotide is complementary to at least a part of a SCN2A mRNA if the sequence is substantially complementary to a non-interrupted portion of an mRNA encoding SCN2A.

By “reducing the activity of SCN2A” is meant decreasing the level of an activity related to SCN2A protein or mutated SCN2A protein (e.g., an ion channel function). The activity level of SCN2A or mutated SCN2A may be measured using any method known in the art (e.g., using standard biophysical methods).

By “reducing the level of SCN2A” is meant decreasing the amount of SCN2A or mutated SCN2A in a cell or subject, e.g., by administering an inhibitor to the cell or subject. The level of SCN2A or mutated SCN2A may be measured using any method known in the art (e.g., by measuring the levels of SCN2A mRNA or levels of SCN2A or mutated SCN2A protein in a cell or a subject). In some embodiments, the decreased amount of SCN2A or mutated SCN2A is assessed relative to the amount of SCN2A or mutated SCN2A prior to or without administering an inhibitor to the cell or subject. In some embodiments, reducing the level of SCN2A is reducing the level of a mutated SCN2A, such as SCN2A comprising R853Q and/or R1882Q.

As used herein, the terms “effective amount,” “therapeutically effective amount,” and “sufficient amount” of an inhibitor that reduces or an enhancer that increases the level and/or activity of SCN2A or mutated SCN2A (e.g., in a cell or a subject) described herein refer to a quantity sufficient to, when administered to the subject, including a human, effect beneficial or desired results, including clinical results, and, as such, an “effective amount” or synonym thereto depends on the context in which it is being applied. For example, in the context of treating a SCN2A-related disorder caused by a gain-of-function mutation, it is an amount of the inhibitor that reduces the level and/or activity of SCN2A or mutated SCN2A sufficient to achieve a treatment response, such as a decrease in one or more symptoms, as compared to the response obtained without administration of the inhibitor that reduces the level and/or activity of SCN2A or mutated SCN2A. Further, in the context of treating a SCN2A-related disorder caused by a loss-of-function mutation, it is an amount of the enhancer that increases the level and/or activity of SCN2A or mutated SCN2A sufficient to achieve a treatment response, such as a decrease in one or more symptoms, as compared to the response obtained without administration of the enhancer that increases the level and/or activity of SCN2A or mutated SCN2A. The amount of a given inhibitor that reduces or enhancer that increases the level and/or activity of SCN2A or mutated SCN2A described herein that will correspond to an effective amount will vary depending upon various factors, such as the given agent, the pharmaceutical formulation, the route of administration, the type of disease or disorder, the identity of the subject (e.g., age, sex, and/or weight) or host being treated, and the like.

As used herein, in some variations, a “therapeutically effective amount” of an inhibitor that reduces or enhancer that increases the level and/or activity of SCN2A or mutated SCN2A of the present disclosure is an amount which results in a beneficial or desired result in a subject as compared to a control. As defined herein, a therapeutically effective amount of an inhibitor that reduces or enhancer that increases the level and/or activity of SCN2A or mutated SCN2A of the present disclosure may be readily determined by one of ordinary skill by routine methods known in the art. Dosage regimen may be adjusted to provide the optimum therapeutic response. “Prophylactically effective amount,” as used herein, is intended to include the amount of a SCN2A inhibitor or SCN2A enhancer that, when administered to a subject having or predisposed to having a SCN2A-related disorder, is sufficient to prevent or ameliorate the disease or one or more symptoms of the disease. Ameliorating the disease includes slowing the course of the disease or reducing the severity of later-developing disease. The “prophylactically effective amount” may vary depending on the SCN2A inhibitor or enhancer, how the agent is administered, the degree of risk of disease, and the history, age, weight, family history, genetic makeup, the types of preceding or concomitant treatments, if any, and other individual characteristics of the subject to be treated. A prophylactically effective amount may also refer to an amount of the inhibitor that reduces or enhancer that increases the level and/or activity of SCN2A or mutated SCN2A (e.g., in a cell or a subject) described herein in a quantity sufficient to, when administered to the subject, including a human, delay the onset of a SCN2A-related disorder, as described herein, by at least 120 days, for example, at least 6 months, at least 12 months, at least 2 years, at least 3 years, at least 4 years, at least 5 years, at least 10 years or more, when compared with the predicted onset.

A “therapeutically effective amount” or “prophylactically effective amount” also includes an amount (either administered in a single or in multiple doses) of a SCN2A inhibitor or enhancer that produces some desired local or systemic effect at a reasonable benefit/risk ratio applicable to any treatment. SCN2A inhibitor or enhancers employed in the methods disclosed herein may be administered in a sufficient amount to produce a reasonable benefit/risk ratio applicable to such treatment.

As used herein, “SCN2A-related disorder” refers to a class of neurological genetic diseases or disorders characterized by aberrant function of SCN2A. SCN2A-related disorders include, for example, epilepsy, pediatric epilepsy, benign familial neonatal/infantile seizures, severe early-onset epilepsy, epileptic encephalopathy, early infantile epileptic encephalopathy (i.e., early onset epileptic encephalopathy), late seizure onset epileptic encephalopathy, Ohtahara syndrome, infantile spasm syndrome (i.e., West syndrome), Lennox-Gastaut syndrome, generalized epilepsy with febrile seizures, migrating partial epilepsy of infancy (i.e., epilepsy of infancy with migrating focal seizures), infantile spasms, autism spectrum disorder, movement disorder, and drug-resistant epilepsies. A SCN2A-related disorder may be caused by a gain-of-function SCN2A mutation. SCN2A-related disorders that may be caused by a gain-of-function SCN2A mutation include, for example, Ohtahara syndrome, epilepsy of infancy with migrating focal seizures, or early onset epileptic encephalopathy. A SCN2A-related disorder may also be caused by a loss-of-function SCN2A mutation. SCN2A-related disorders that may be caused by a loss-of-function SCN2A mutation include, for example, autism spectrum disorder, benign familial neonatal/infantile seizures, infantile spasms, late seizure onset epileptic encephalopathy, or epilepsy of infancy with migrating focal seizures. In certain embodiments, a SCN2A-related disorder may be caused by a combination of both a gain-of-function mutation and a loss-of-function mutation. In certain embodiments, one allele of the gene contains a gain-of-function or loss-of-function mutation, and in certain embodiments, both alleles of the gene contain a gain-of-function and/or loss-of-function mutation.

As used herein, the term “subject” refers to any organism to which a composition in accordance with embodiments disclosed herein may be administered, e.g., for experimental, diagnostic, prophylactic, and/or therapeutic purposes. Typical subjects include any animal (e.g., mammals such as mice, rats, rabbits, non-human primates, and humans). A subject may seek or be in need of treatment, require treatment, be receiving treatment, be receiving treatment in the future, or be a human or animal who is under care by a trained professional for a particular disease or condition. In particular embodiments, a “subject” is a pediatric subject. A pediatric subject is a child under the age of 18 years, such as under the age of 17 years, under the age of 16 years, under the age of 15 years, under the age of 14 years, under the age of 13 years, under the age of 12 years, under the age of 11 years, under the age of 10 years, under the age of 9 years, under the age of 8 years, under the age of 7 years, under the age of 6 years, under the age of 5 years, under the age of 4 years, under the age of 3 years, under the age of 2 years, under the age of 1 year, under the age of 6 months, under the age of 3 months or under the age of 1 month. Typically, it is known that the subject has a SCN2A mutation prior to administration of the SCN2A inhibitor or SCN2A enhancer, however, the status of the SCN2A mutation as a gain-of-function or loss-of-function mutation has not been verified by genetic or biochemical (e.g., patch clamp) testing.

As used herein, the terms “treat,” “treated,” or “treating” mean both therapeutic treatment and prophylactic or preventative measures wherein the object is to prevent or slow down (lessen) an undesired physiological condition, disorder, or disease, or obtain beneficial or desired clinical results. Beneficial or desired clinical results include, but are not limited to, alleviation of symptoms; diminishment of the extent of a condition, disorder, or disease; stabilized (i.e., not worsening) state of the condition, disorder, or disease; delay in onset or slowing of the condition, disorder, or disease progression; amelioration of the condition, disorder, or disease state or remission (whether partial or total), whether detectable or undetectable; an amelioration of at least one measurable physical parameter, not necessarily discernible by the subject; and enhancement or improvement of the condition, disorder, or disease. Treatment includes eliciting a clinically-significant response without excessive levels of side effects. Treatment also includes prolonging survival as compared to expected survival if not receiving treatment.

The details of one or more embodiments are set forth in the description below. Other features, objects, and advantages of the embodiments disclosed herein will be apparent from the description and from the claims.

### SCN2A-Related Disorders

Disclosed herein are compositions and methods to treat SCN2A-related disorders in a subject. In some embodiments, the subject has a gain-of-function SCN2A mutation and a SCN2A inhibitor is used to reduce SCN2A levels or activity. As discussed herein, mutations in SCN2A may be gain-of-function mutations or loss-of-function mutations that may be associated with various disorders including BFNIS, autism, including autism with and without epilepsy, schizophrenia, intellectual disability, infantile spasms, and epileptic encephalopathies including Ohtahara syndrome, EIMFS, late seizure onset epileptic encephalopathy (e.g., LOE), and EOEE.

The SCN2A gene encodes for a SCN2A mRNA transcript, which further encodes for a SCN2A protein. In various embodiments, mutation of SCN2A leads to a mutated SCN2A mRNA transcript and/or a mutated SCN2A protein. In some embodiments, a mutated SCN2A mRNA transcript is translated into a mutated SCN2A protein with gain-of-function properties. A mutated SCN2A mRNA transcript may also be translated into a mutated SCN2A protein with a loss-of-function properties.

Example accession numbers for human SCN2A mRNA transcript and human SCN2A protein are detailed below:
SCN2A mRNA transcript: NM_001040142.2, NM_001040143.2, NM_001371246.1, NM_001371247.1, and NM_021007.3 (SEQ ID NO: 2); and
SCN2A protein: NP_001035232.1, NP_001035233.1, and NP_066287.2 (SEQ ID NO: 1).

In certain embodiments, a SCN2A inhibitor, such as a SCN2A nucleic acid inhibitor molecule (e.g., an ASO) is administered to a subject in an amount effective to reduce the level and/or activity of a SCN2A protein. In various embodiments, the SCN2A inhibitor reduces the level and/or activity of a SCN2A protein by targeting a SCN2A mRNA transcript that encodes for the SCN2A protein. In various embodiments, the SCN2A inhibitor reduces the level and/or activity of a SCN2A protein encoded by a SCN2A mRNA transcript having a sequence sharing at least 80% sequence identity with an equal length portion of any one of NM_001040142.2, NM_001040143.2, NM_001371246.1, NM_001371247.1, or NM_021007.3 (SEQ ID NO: 2). In various embodiments, the SCN2A inhibitor reduces the level and/or activity of a SCN2A protein encoded by a SCN2A mRNA transcript having a sequence sharing at least 81% identity, at least 82% identity, at least 83% identity, at least 84% identity, at least 85% identity, at least 86% identity, at least 87% identity, at least 88% identity, at least 89% identity, at least 90% identity, at least 91% identity, at least 92% identity, at least 93% identity, at least 94% identity, at least 95% identity, at least 96% identity, at least 97% identity, at least 98% identity, or at least 99% identity with an equal length portion of any one of NM_001040142.2, NM_001040143.2, NM_001371246.1, NM_001371247.1, or NM_021007.3 (SEQ ID NO: 2). In some embodiments, the SCN2A inhibitor (e.g., a nucleic acid inhibitor molecule, such as a SCN2A antisense oligonucleotide) reduces the level and/or activity of a SCN2A protein having one or more gain-of-function mutations.

In various embodiments, the SCN2A protein that is inhibited, such as reduced in expression level, by the SCN2A inhibitor includes an amino acid sequence that shares at least 80% sequence identity with an equal length portion of any one of NP_001035232.1, NP_001035233.1, or NP_066287.2 (SEQ ID NO: 1). In various embodiments, the SCN2A protein that is inhibited by the SCN2A inhibitor includes an amino acid sequence that shares at least 81% identity, at least 82% identity, at least 83% identity, at least 84% identity, at least 85% identity, at least 86% identity, at least 87% identity, at least 88% identity, at least 89% identity, at least 90% identity, at least 91% identity, at least 92% identity, at least 93% identity, at least 94% identity, at least 95% identity, at least 96% identity, at least 97% identity, at least 98% identity, or at least 99% identity with an equal length portion of any one of NP_001035232.1, NP_001035233.1, or NP_066287.2 (SEQ ID NO: 1). In some embodiments, the SCN2A protein that is inhibited by the SCN2A inhibitor (e.g., a nucleic acid inhibitor molecule, such as a SCN2A antisense oligonucleotide) has one or more mutations that are gain-of-function mutations.

In other embodiments, a SCN2A enhancer, such as a SCN2A nucleic acid inhibitor molecule (e.g., an ASO) is administered to a subject in an amount effective to increase the level and/or activity of a SCN2A protein. In various embodiments, the SCN2A enhancer increases the level and/or activity of a SCN2A protein by targeting a SCN2A mRNA transcript that encodes for the SCN2A protein. In various embodiments, the SCN2A enhancer increases the level and/or activity of a SCN2A protein encoded by a SCN2A mRNA transcript having a sequence that shares at least 80% sequence identity with an equal length portion of any one of NM_001040142.2, NM_001040143.2, NM_001371246.1, NM_001371247.1, or NM_021007.3 (SEQ ID NO: 2). In various embodiments, the SCN2A enhancer increases the level and/or activity of a SCN2A protein encoded by a SCN2A mRNA transcript having a sequence that shares at least 81% identity, at least 82% identity, at least 83% identity, at least 84% identity, at least 85% identity, at least 86% identity, at least 87% identity, at least 88% identity, at least 89% identity, at least 90% identity, at least 91% identity, at least 92% identity, at least 93% identity, at least 94% identity, at least 95% identity, at least 96% identity, at least 97% identity, at least 98% identity, or at least 99% identity with an equal length portion of any one of NM_001040142.2, NM_001040143.2, NM_001371246.1, NM_001371247.1, or NM_021007.3 (SEQ ID NO: 2). In some embodiments, the SCN2A enhancer (e.g., a SCN2A antisense oligonucleotide) increases the level and/or activity of a SCN2A protein having one or more loss-of-function mutations.

In various embodiments, the SCN2A protein that is enhanced, such as by an increase in expression level, by the SCN2A enhancer includes an amino acid sequence that shares at least 80% sequence identity with an equal length portion of any one of NP_001035232.1, NP_001035233.1, or NP_066287.2 (SEQ ID NO: 1). In various embodiments, the SCN2A protein that is enhanced by the SCN2A enhancer includes an amino acid sequence that shares at least 81% identity, at least 82% identity, at least 83% identity, at least 84% identity, at least 85% identity, at least 86% identity, at least 87% identity, at least 88% identity, at least 89% identity, at least 90% identity, at least 91% identity, at least 92% identity, at least 93% identity, at least 94% identity, at least 95% identity, at least 96% identity, at least 97% identity, at least 98% identity, or at least 99% identity with an equal length portion of any one of NP_001035232.1, NP_001035233.1, or NP_066287.2 (SEQ ID NO: 1). In some embodiments, the SCN2A protein that is enhanced by the SCN2A enhancer, such as a SCN2A antisense oligonucleotide, has one or more loss-of-function mutations.

### SCN2A Inhibitors and SCN2A Enhancers

SCN2A inhibitors or SCN2A enhancers described herein that reduce or increase, respectively, the level and/or activity of SCN2A or mutated SCN2A in a cell or subject may be, for example, a polynucleotide, e.g., an oligonucleotide. These SCN2A inhibitors or enhancers reduce or increase, respectively, the level of an activity related to SCN2A or mutated SCN2A, or a related downstream effect, or reduce or increase, respectively, the level of SCN2A or mutated SCN2A in a cell or subject. In various embodiments, an SCN2A inhibitor or enhancer effective for treating a SCN2A-related disorder is nucleic acid inhibitor molecule.

In certain embodiments, the nucleic acid inhibitor molecule is a double-stranded RNAi inhibitor molecule comprising a sense (or passenger) strand and an antisense (or guide strand). A variety of double stranded RNAi inhibitor molecule structures are known in the art. For example, early work on RNAi inhibitor molecules focused on double-stranded nucleic acid molecules with each strand having sizes of 19-25 nucleotides with at least one 3′-overhang of 1 to 5 nucleotides (see, e.g., U.S. Pat. No. 8,372,968, incorporated by reference herein). Subsequently, longer double-stranded RNAi inhibitor molecules that get processed in vivo by the Dicer enzyme to active RNAi inhibitor molecules were developed (see, e.g., U.S. Pat. No. 8,883,996, incorporated by reference herein). Later work developed extended double-stranded nucleic acid inhibitor molecules where at least one end of at least one strand is extended beyond the double-stranded targeting region of the molecule, including structures where one of the strands includes a thermodynamically-stabilizing tetraloop structure (see, e.g., U.S. Pat. Nos. 8,513,207, 8,927,705, WO 2010/033225, and WO 2016/100401, incorporated by reference herein). Those structures include single-stranded extensions (on one or both sides of the molecule) and double-stranded extensions.

In certain embodiments, the nucleic acid inhibitor molecule is a single-stranded nucleic acid inhibitor molecule. Single stranded nucleic acid inhibitor molecules are known in the art. For example, recent efforts have demonstrated activity of single-stranded RNAi inhibitor (ssRNAi) molecules (see, e.g., Matsui et al., Molecular Therapy, 2016, 24(5):946-55. Single-stranded antisense oligonucleotides have been used for decades to reduce expression of specific target genes. Pelechano and Steinmetz, Nature Review Genetics, 2013, 14:880-93. A number of variations on the common themes of these structures have been developed for a range of targets. Single stranded nucleic acid inhibitor molecules include, for example, conventional antisense oligonucleotides (ASOs), microRNA, ribozymes, aptamers, antagomirs, and ssRNAi inhibitor molecules, all of which are known in the art.

The nucleic acid inhibitor molecules, including ASOs, can be designed to be complementary to a target sequence, such as a target sequence of a SCN2A mRNA transcript or SCN2A mRNA transcript variant. As an additional example, nucleic acid inhibitor molecules, including ASOs, can be an enhancer designed to be complementary to a target sequence of a naturally occurring SCN2A antisense transcript that reduces activity of an SCN2A gene. As yet another example, nucleic acid inhibitor molecules, including ASOs, can be an enhancer designed to be complementary to a target sequence of an open reading frame that is upstream (a uORF) of the SCN2A open reading frame that increases the level of SCN2A protein by enhancing translation of the SCN2A open reading frame. Nucleic acid inhibitor molecules can decrease the expression level (e.g., protein level or mRNA level) of SCN2A or a mutated SCN2A. In some embodiments, the nucleic acid inhibitor molecule, such as an ASO, targets a coding sequence. In some embodiments, the nucleic acid inhibitor molecules, such as an ASO, targets a non-coding sequence. In some embodiments, the ASO that targets a non-coding sequence is an enhancer that stabilizes the mRNA, thereby increasing the level of SCN2A protein. In certain embodiments, the SCN2A inhibitor is an ASO disclosed, for example, in PCT Publication No. WO 2020/041348 and PCT Application No. PCT/US2021/044887, published as WO 2022/032060, each of which is incorporated by reference herein.

In some embodiments, the nucleic acid inhibitor molecule is a single-stranded antisense oligonucleotide (ASO). Oligonucleotides include DNA and DNA/RNA chimeric molecules, typically about 10 to 30 nucleotides in length, which recognize polynucleotide target sequences or sequence portions through hydrogen bonding interactions with the nucleotide bases of the target sequence (e.g., SCN2A mRNA transcript or SCN2A mRNA transcript variant).

In various embodiments, the nucleic acid inhibitor molecule, such as the ASO, may be at least 8 nucleobases in length. In various embodiments, the nucleic acid inhibitor molecule, such as the ASO, may be at least 9, at least 10, at least 11, at least 12, at least 13, at least 14, at least 15, at least 16, at least 17, at least 18, at least 19, at least 20, at least 21, at least 22, at least 23, at least 24, or at least 25 nucleobases in length. In various embodiments, the nucleic acid inhibitor molecule, such as the ASO, is 8 nucleobases in length. In various embodiments, the nucleic acid inhibitor molecule, such as the ASO, is 9 nucleobases in length, 10 nucleobases in length, 11 nucleobases in length, 12 nucleobases in length, 13 nucleobases in length, 14 nucleobases in length, 15 nucleobases in length, 16 nucleobases in length, 17 nucleobases in length, 18 nucleobases in length, 19 nucleobases in length, 20 nucleobases in length, 21 nucleobases in length, 22 nucleobases in length, 23 nucleobases in length, 24 nucleobases in length, or 25 nucleobases in length. In various embodiments, the nucleic acid inhibitor molecule, such as the ASO, may be between 17-22, 18-21, or 19-20 nucleobases in length. In some embodiments, the nucleic acid inhibitor molecule, such as the ASO, is at least 9 nucleobases in length, such as at least any of 9, 10, 11, 12, 13, 14, 15, 16, 17, 18, 19, 20, 21, 22, 23, 24, 25, 25, 26, 27, 28, 29, or 30 nucleobases in length. In some embodiments, the nucleic acid inhibitor molecule, such as the ASO, is any of 8, 9, 10, 11, 12, 13, 14, 15, 16, 17, 18, 19, 20, 21, 22, 23, 24, 25, 26, 27, 28, 29, or 30 nucleobases in length. In some embodiments, the nucleic acid inhibitor molecule, such as the ASO, is between any of 17-22, 18-21, or 19-20 nucleobases in length.

In various embodiments, an oligonucleotide refers to a short polynucleotide (e.g., a polynucleotide of 100 or fewer linked nucleosides). The certain embodiments, the nucleic acid inhibitor molecule is a double-stranded nucleic acid inhibitor molecule comprising about 10-30 base pairs in length or about 18-22 (e.g., 18-20) base pairs in length, for example, about 10, 11, 12, 13, 14, 15, 16, 17, 18, 19, 20, 21, 22, 23, 24, 25, 26, 27, 28, 29, or 30 base pairs in length, such as about 15-30, 15-29, 15-28, 15-27, 15-26, 15-25, 15-24, 15-23, 15-22, 15-21, 15-20, 15-19, 15-18, 15-17, 18-30, 18-29, 18-28, 18-27, 18-26, 18-25, 18-24, 18-23, 18-22, 18-21, 18-20, 19-30, 19-29, 19-28, 19-27, 19-26, 19-25, 19-24, 19-23, 19-22, 19-21, 19-20, 20-30, 20-29, 20-28, 20-27, 20-26, 20-25, 20-24, 20-23, 20-22, 20-21, 21-30, 21-29, 21-28, 21-27, 21-26, 21-25, 21-24, 21-23, or 21-22 base pairs in length. In some embodiments, the double-stranded nucleic acid inhibitor molecule may be any length, for example, about any of 10, 11, 12, 13, 14, 15, 16, 17, 18, 19, 20, 21, 22, 23, 24, 25, 26, 27, 28, 29, or 30 base pairs in length. In some embodiments, the double-stranded nucleic acid inhibitor molecule is any of about 15-30, 15-29, 15-28, 15-27, 15-26, 15-25, 15-24, 15-23, 15-22, 15-21, 15-20, 15-19, 15-18, 15-17, 18-30, 18-29, 18-28, 18-27, 18-26, 18-25, 18-24, 18-23, 18-22, 18-21, 18-20, 19-30, 19-29, 19-28, 19-27, 19-26, 19-25, 19-24, 19-23, 19-22, 19-21, 19-20, 20-30, 20-29, 20-28, 20-27, 20-26, 20-25, 20-24, 20-23, 20-22, 20-21, 21-30, 21-29, 21-28, 21-27, 21-26, 21-25, 21-24, 21-23, or 21-22 base pairs in length.

Ranges and lengths intermediate to the above recited ranges and lengths are also contemplated to be part of the disclosed embodiments.

In some embodiments, the nucleic acid inhibitor molecule, such as the ASO, is an inhibitor that decreases the level and/or activity or function of SCN2A or mutated SCN2A. In some embodiments, the nucleic acid inhibitor molecule, such as the ASO, inhibits expression, such as protein expression, of SCN2A or mutated SCN2A. In other embodiments, the nucleic acid inhibitor molecule, such as the ASO, increases degradation of SCN2A or mutated SCN2A protein or mRNA transcripts and/or decreases the stability (e.g., half-life) of SCN2A or mutated SCN2A protein or mRNA transcripts.

In some embodiments, the nucleic acid inhibitor molecule, such as the ASO, is an enhancer that increases the level and/or activity or function of SCN2A. In some embodiments, the nucleic acid inhibitor molecule, such as the ASO, enhances expression, such as protein expression, of SCN2A such as by blocking an uORF. In other embodiments, the nucleic acid inhibitor molecule, such as the ASO, reduces degradation of SCN2A or mRNA transcripts and/or increases the stability (e.g., half-life) of SCN2A or mutated SCN2A protein or mRNA transcripts, such as by binding to non-translated regions of the mRNA transcript.

In some embodiments, the nucleic acid inhibitor molecule, such as the ASO, includes a region of complementarity (e.g., a contiguous nucleobase region) which is complementary to at least a part of an mRNA transcribed in the expression of a SCN2A gene. In some embodiments, the region of complementarity may be about 30 nucleotides or less in length (e.g., about any of 30, 29, 28, 27, 26, 25, 24, 23, 22, 21, 20, 19, 18, 17, 16, 15, 14, 13, 12, 11, or 10 nucleotides or less in length).

In some embodiments, the region of complementarity to a target sequence (e.g., target sequence of a SCN2A mRNA transcript) may be between 10 and 30 linked nucleosides in length, e.g., between any of 10-29, 10-28, 10-27, 10-26, 10-25, 10-24, 10-23, 10-22, 10-21, 10-20, 10-19, 10-18, 10-17, 10-16, 10-15, 10-14, 10-13, 10-12, 15-29, 15-28, 15-27, 15-26, 15-25, 15-24, 15-23, 15-22, 15-21, 15-20, 15-19, 15-18, 15-17, 18-30, 18-29, 18-28, 18-27, 18-26, 18-25, 18-24, 18-23, 18-22, 18-21, 18-20, 19-30, 19-29, 19-28, 19-27, 19-26, 19-25, 19-24, 19-23, 19-22, 19-21, 19-20, 20-30, 20-29, 20-28, 20-27, 20-26, 20-25, 20-24, 20-23, 20-22, 20-21, 21-30, 21-29, 21-28, 21-27, 21-26, 21-25, 21-24, 21-23, or 21-22 linked nucleosides in length. Ranges and lengths intermediate to the above recited ranges and lengths are also contemplated to be part of the invention.

In some embodiments, the oligonucleotide is an ASO capable of hybridizing to a portion of SCN2A RNA (e.g., mRNA), namely, a SCN2A ASO. In some embodiments, the SCN2A ASO is capable of hybridizing, such as is complementary to a portion of SCN2A mRNA, e.g., fully complementary, to SCN2A or a mutant thereof. In some embodiments, the SCN2A ASO is a gapmer (an oligonucleotide comprising a central DNA portion flanked by RNA portions, wherein in some embodiments the RNA portions comprise modified RNA). In some embodiments, the nucleic acid inhibitor molecule is a small interfering RNA (siRNA). In some embodiments, the nucleic acid inhibitor molecule is a microRNA (miRNA).

In some embodiments, the nucleic acid inhibitor molecule, such as SCN2A ASO, comprises one or more modified sugars. In some embodiments, each of the one or more modified sugars is independently selected from the group consisting of a bicyclic sugar, a 2′-O-methoxyethyl (2MOE) modified sugar, a 2′-O-methyl (2-OMe) modified sugar, a 2′-methoxy modified sugar, a 2′-Fluoro modified sugar, a 2′-O-alkyl modified sugar, a constrained ethyl (cEt) modified sugar, a locked sugar, and an unlocked sugar.

In some embodiments, the nucleic acid inhibitor molecule, such as SCN2A ASO, includes one or more modified internucleoside linkages. In some embodiments, one or more of the modified internucleoside linkages includes a modified phosphate. In some embodiments, each of the modified phosphates is independently selected from the group consisting of a phosphodiester, phosphorothioate, a phosphorodithioate, a phosphoramidate, a phosphorodiamidate, a thiophosphoramidate, a thiophosphorodiamidate, a methyl phosphonate, a phosphoromorpholidate, and a phosphoropiperazidate.

In some embodiments, the nucleic acid inhibitor molecule, such as SCN2A ASO, includes one or more modified nucleobases. In some embodiments, the modified nucleobase is selected from the group consisting of 5-methylcytosine, 5-hydroxymethyl cytosine, xanthine, hypoxanthine, 2-aminoadenine, 6-methyladenine, 6-methylguanine, 2-propyladenine, 2-propylguanine, 2-thiouracil, 2-thiothymine, 2-thiocytosine, 5-halouracil, 5-halocytosine, 5-propynyluracil, 5-propynylcytosine, 6-azouracil, 6-azocytosine, 6-azothymine, 5-uracil (pseudouracil), 4-thiouracil, 8-haloadenine, 8-aminoadenine, 8-thioladenine, 8-thioalkyladenine, 8-hydroxyladenine, 8-haloguanine, 8-aminoguanine, 8-thiolguanine, 8-thioalkylguanine, 8-hydroxylguanine, 5-bromouracil, 5-trifluoromethyluracil, 5-bromocytosine, 5-trifluoromethylcytosine, 7-methylguanine, 7-methyladenine, 2-fluoroadenine, 8-azaguanine, 8-azaadenine, 7-deazaguanine, 7-deazaadenine, 3-deazaguanine, and 3-deazaadenine.

In some embodiments, the nucleic acid inhibitor molecule, such as SCN2A ASO, comprises one or more modified ribonucleotides. In some embodiments, the modified ribonucleotide is a cytidine deoxynucleoside with a heterocyclic 5-methyl group. In some embodiments, the modified ribonucleotide is a 2′-O-methoxyethyl ribonucleotide. In some embodiments, the modified ribonucleotide is a cytidine 2′-O-methoxyethyl ribonucleotides with heterocyclic 5-methyl groups. In some embodiments, the modified ribonucleotide is uridine 2′-O-methoxyethyl ribonucleotides with heterocyclic 5-methyl groups.

In some embodiments, the nucleic acid inhibitor molecule, such as the ASO, can be synthesized by standard methods known in the art and as further discussed below, e.g., by use of an automated DNA synthesizer, such as are commercially available from, for example, Biosearch, Applied Biosystems, Inc.

In some embodiments, the nucleic acid inhibitor molecule, such as the ASO, can be prepared using solution-phase or solid-phase organic synthesis or both. Organic synthesis offers the advantage that the oligonucleotide comprising unnatural or alternative nucleotides can be easily prepared. Single-stranded nucleic acid inhibitor molecule, such as ASOs, disclosed herein can be prepared using solution-phase or solid-phase organic synthesis or both.

Inhibitor and enhancer oligonucleotides can be designed by methods well known in the art. While a target sequence is generally about 10-30 linked nucleosides in length, there is wide variation in the suitability of particular sequences in this range for directing cleavage of any given target RNA.

Oligonucleotides with homology sufficient to provide sequence specificity required to uniquely degrade any RNA can be designed using programs known in the art.

Systematic testing of several designed species for optimization of the inhibitor and enhancer oligonucleotide sequence can also be undertaken in accordance with the teachings provided herein. Considerations when designing inhibitor or enhancer oligonucleotides include, but are not limited to, biophysical, thermodynamic, and structural considerations, base preferences at specific positions, and homology. The making and use of therapeutic agents based on non-coding oligonucleotides are also known in the art.

The nucleic acid inhibitor molecules may also comprise or be generated from double-stranded small interfering RNAs (siRNAs), in which sequences fully complementary to cellular nucleic acid sequences (e.g., mRNAs) mediate degradation or in which sequences incompletely complementary to cellular nucleic acid sequences (e.g., mRNAs) mediate translational repression when expressed within cells. In another embodiment, double-stranded siRNAs can be processed into single-stranded antisense RNAs that bind single-stranded cellular RNAs (e.g., microRNAs) and inhibit their expression. RNA interference (RNAi) is the process of sequence-specific, post-transcriptional gene silencing in animals and plants, initiated by double-stranded RNA (dsRNA) that is homologous in sequence to the silenced gene. In vivo, long dsRNA is cleaved by ribonuclease III to generate 21- and 22-nucleotide siRNAs. It has been shown that 21-nucleotide siRNA duplexes specifically suppress expression of endogenous and heterologous genes in different mammalian cell lines, including human embryonic kidney (HEK293) and HeLa cells (Elbashir et al. (2001) Nature 411:494-498). Accordingly, translation of a gene in a cell can be inhibited by contacting the cell with short double-stranded RNAs having a length of about 15 to 30 nucleotides or of about 18 to 21 nucleotides or of about 19 to 21 nucleotides. Alternatively, a vector encoding for such siRNAs or short hairpin RNAs (shRNAs) that are metabolized into siRNAs can be introduced into a target cell (see, e.g., McManus et al. (2002) RNA 8:842; Xia et al. (2002) Nature Biotechnology 20:1006; and Brummelkamp et al. (2002) Science 296:550). Vectors that can be used are commercially available, e.g., from OligoEngine under the name pSuper RNAi System™.

The nucleic acid inhibitor molecules disclosed herein may also comprise ribozyme molecules designed to catalytically cleave cellular mRNA transcripts, which can also be used to prevent translation of cellular mRNAs and expression of cellular polypeptides, or both (See, e.g., PCT International Publication WO90/11364, published Oct. 4, 1990; Sarver et al. (1990) Science 247:1222-1225, and U.S. Pat. No. 5,093,246). While ribozymes that cleave mRNA at site specific recognition sequences can be used to destroy cellular mRNAs, the use of hammerhead ribozymes is preferred. Hammerhead ribozymes cleave mRNAs at locations dictated by flanking regions that form complementary base pairs with the target mRNA. The sole requirement is that the target mRNA have the following sequence of two bases: 5′-UG-3′. The construction and production of hammerhead ribozymes is well-known in the art and is described more fully in Haseloff and Gerlach (1988) Nature 334:585-591. The ribozyme may be engineered so that the cleavage recognition site is located near the 5′ end of cellular mRNAs, i.e., to increase efficiency and minimize the intracellular accumulation of non-functional mRNA transcripts.

The ribozymes of the methods presented herein also include RNA endoribonucleases (hereinafter “Cech-type ribozymes”), such as the one which occurs naturally in Tetrahymena thermophila (known as the IVS or L-19 IVS RNA) and which has been extensively described by Thomas Cech and collaborators (See, e.g., Zaug et al. (1984) Science 224:574-578; Zaug et al. (1986) Science 231:470-475; Zaug et al. (1986) Nature 324:429-433; published International patent application No. WO88/04300 by University Patents Inc.; and Been et al. (1986) Cell 47:207-216). The Cech-type ribozymes have an eight base pair active site that hybridizes to a target RNA sequence whereafter cleavage of the target RNA takes place. The methods and compositions presented herein encompass those Cech-type ribozymes that target eight base-pair active site sequences that are present in cellular genes.

As in the antisense approach, the ribozymes can be composed of modified oligonucleotides (e.g., for improved stability, targeting, etc.). A preferred method of delivery involves using a DNA construct “encoding” the ribozyme under the control of a strong constitutive pol III or pol II promoter, so that transfected cells will produce sufficient quantities of the ribozyme to destroy endogenous cellular messages and inhibit translation. Because ribozymes, unlike antisense molecules, are catalytic, a lower intracellular concentration is required for efficiency.

### Additional Inhibitors and Enhancers

Additional inhibitors and enhancers useful in the methods disclosed herein include small molecules, peptides, and peptidomimetics that can bind and/or modulate SCN2A. In particular, the inhibitors and enhancers include small molecule compounds which can inhibit or enhance the activity of SCN2A. The small molecule compounds can be obtained using any of the numerous approaches in combinatorial library methods known in the art, including: spatially addressable parallel solid phase or solution phase libraries; synthetic library methods requiring deconvolution; the “one-bead one-compound” library method; and synthetic library methods using affinity chromatography selection. (Lam, K. S. (1997) Anticancer Drug Des. 12:145).

Examples of methods for the synthesis of molecular libraries of small molecule compounds can be found in the art, for example in: DeWitt et al. (1993) Proc. Natl. Acad. Sci. USA 90:6909; Erb et al. (1994) Proc. Natl. Acad. Sci. USA 91:11422; Zuckermann et al. (1994) J. Med. Chem. 37:2678; Cho et al. (1993) Science 261:1303; Carrell et al. (1994) Angew. Chem. Int. Ed. Engl. 33:2059; Carell et al. (1994) Angew. Chem. Int. Ed. Engl. 33:2061; and in Gallop et al. (1994) J. Med. Chem. 37:1233.

Libraries of small molecule compounds can be presented in solution (e.g., Houghten (1992) Biotechniques 13:412-421), or on beads (Lam (1991) Nature 354:82-84), chips (Fodor (1993) Nature 364:555-556), bacteria (Ladner U.S. Pat. No. 5,223,409), spores (Ladner supra), plasmids (Cull et al. (1992) Proc. Natl. Acad. Sci. USA 89:1865-1869) or on phage (Scott and Smith (1990) Science 249:386-390); (Devlin (1990) Science 249:404-406); (Cwirla et al. (1990) Proc. Natl. Acad. Sci. USA 87:6378-6382); (Felici (1991) J. Mol. Biol. 222:301-310); (Ladner supra.). Small molecule compounds can be screened in cell based or non-cell based assays. Small molecule compounds can be screened in pools (e.g., multiple compounds in each testing sample) or as individual compounds.

### Pharmaceutical Compositions

The term “pharmaceutical composition,” as used herein, represents a composition containing a compound described herein formulated with a pharmaceutically acceptable excipient, and preferably manufactured or sold with the approval of a governmental regulatory agency as part of a therapeutic regimen for the treatment of disease in a mammal. Pharmaceutical compositions can be formulated, for example, for oral administration in unit dosage form (e.g., a tablet, capsule, caplet, gelcap, or syrup); for topical administration (e.g., as a cream, gel, lotion, or ointment); for intravenous administration (e.g., as a sterile solution free of particulate emboli and in a solvent system suitable for intravenous use); for intrathecal injection; for intracerebroventricular injections; for intraparenchymal injection; or in any other pharmaceutically acceptable formulation.

A “pharmaceutically acceptable excipient,” as used herein, refers to any ingredient other than the compounds described herein (for example, a vehicle capable of suspending or dissolving the active compound) and having the properties of being substantially nontoxic and non-inflammatory in a subject. Excipients may include, for example: antiadherents, antioxidants, binders, coatings, compression aids, disintegrants, dyes (colors), emollients, emulsifiers, fillers (diluents), film formers or coatings, flavors, fragrances, glidants (flow enhancers), lubricants, preservatives, printing inks, sorbents, suspending or dispersing agents, sweeteners, and waters of hydration. Exemplary excipients include, but are not limited to: butylated hydroxytoluene (BHT), calcium carbonate, calcium phosphate (dibasic), calcium stearate, croscarmellose, crosslinked polyvinyl pyrrolidone, citric acid, crospovidone, cysteine, ethylcellulose, gelatin, hydroxypropyl cellulose, hydroxypropyl methylcellulose, lactose, magnesium stearate, maltitol, mannitol, methionine, methylcellulose, methyl paraben, microcrystalline cellulose, polyethylene glycol, polyvinyl pyrrolidone, povidone, pregelatinized starch, propyl paraben, retinyl palmitate, shellac, silicon dioxide, sodium carboxymethyl cellulose, sodium citrate, sodium starch glycolate, sorbitol, starch (corn), stearic acid, sucrose, talc, titanium dioxide, vitamin A, vitamin E, vitamin C, and xylitol.

As used herein, the term “pharmaceutically acceptable salt” means any pharmaceutically acceptable salt of any of the compounds described herein. For example, pharmaceutically acceptable salts of any of the compounds described herein include those that are within the scope of sound medical judgment, suitable for use in contact with the tissues of humans and animals without undue toxicity, irritation, and allergic response and are commensurate with a reasonable benefit/risk ratio. Pharmaceutically acceptable salts are well known in the art. For example, pharmaceutically acceptable salts are described in: Berge et al., J. Pharmaceutical Sciences 66:1-19, 1977 and in Pharmaceutical Salts: Properties, Selection, and Use, (Eds. P. H. Stahl and C. G. Wermuth), Wiley-VCH, 2008. The salts can be prepared in situ during the final isolation and purification of the compounds described herein or separately by reacting a free base group with a suitable organic acid.

The compounds described herein may have ionizable groups so as to be capable of preparation as pharmaceutically acceptable salts. These salts may be acid addition salts involving inorganic or organic acids or the salts may, in the case of acidic forms of the compounds described herein, be prepared from inorganic or organic bases. Frequently, the compounds are prepared or used as pharmaceutically acceptable salts prepared as addition products of pharmaceutically acceptable acids or bases. Suitable pharmaceutically acceptable acids and bases and methods for preparation of the appropriate salts are well-known in the art. Salts may be prepared from pharmaceutically acceptable non-toxic acids and bases including inorganic and organic acids and bases. Representative acid addition salts include acetate, adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, fumarate, glucoheptonate, glycerophosphate, hemisulfate, heptonate, hexanoate, hydrobromide, hydrochloride, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, toluenesulfonate, undecanoate, and valerate salts. Representative alkali or alkaline earth metal salts include sodium, lithium, potassium, calcium, and magnesium, as well as nontoxic ammonium, quaternary ammonium, and amine cations, including, but not limited to ammonium, tetramethylammonium, tetraethylammonium, methylamine, dimethylamine, trimethylamine, triethylamine, and ethylamine.

In some embodiments, the inhibitors and enhancers described herein are formulated into pharmaceutical compositions for administration to human subjects in a biologically compatible form suitable for administration in vivo.

The compounds described herein may be used in the form of the free base, or in the form of salts, solvates, or prodrugs. All forms are within the methods described herein. In accordance with the methods disclosed herein, the described compounds or salts, solvates, or prodrugs thereof may be administered to a subject in a variety of forms depending on the selected route of administration, as will be understood by those skilled in the art. The compounds described herein may be administered, for example, by oral, parenteral, intrathecal, intracerebroventricular, intraparenchymal, buccal, sublingual, nasal, rectal, patch, pump, or transdermal administration and the pharmaceutical compositions formulated accordingly. Parenteral administration includes intravenous, intraperitoneal, subcutaneous, intramuscular, transepithelial, nasal, intrapulmonary, intrathecal, intracisternal, intracerebroventricular, intraparenchymal, rectal, and topical modes of administration. Parenteral administration may be by continuous infusion over a selected period of time.

The compounds described herein may be administered to an animal, e.g., a human, alone or in combination with pharmaceutically acceptable carriers, as noted herein, the proportion of which is determined by the solubility and chemical nature of the compound, chosen route of administration, and standard pharmaceutical practice.

Dosages of SCN2A Inhibitors, SCN2A Enhancers and/or Pharmaceutical Compositions

The dosage of the compositions (e.g., a pharmaceutical composition including an SCN2A inhibitor or enhancer, such as a SCN2A ASO) described herein can vary depending on many factors, such as the pharmacodynamic properties of the compound; the mode of administration; the age, health, and weight of the subject; the nature and extent of the symptoms; the frequency of the treatment, and the type of concurrent treatment, if any; and the clearance rate of the compound in the animal to be treated. The compositions described herein may be administered initially in a suitable dosage that may be adjusted as required, depending on the clinical response. In some embodiments, the dosage of a composition (e.g., a composition including an oligonucleotide) is a prophylactically or a therapeutically effective amount.

In various embodiments, the dose of the SCN2A inhibitor or enhancer, such as a SCN2A nucleic acid inhibitor (e.g., ASO), administered to a subject can be at least about 0.1 mg/kg. In various embodiments, the dose of the SCN2A inhibitor or enhancer administered to a subject is at least about 0.2 mg/kg, at least about 0.3 mg/kg, at least about 0.4 mg/kg, at least about 0.5 mg/kg, at least about 0.6 mg/kg, at least about 0.7 mg/kg, at least about 0.8 mg/kg, at least about 0.9 mg/kg, at least about 1.0 mg/kg, at least about 1.1 mg/kg, at least about 1.2 mg/kg, at least about 1.3 mg/kg, at least about 1.4 mg/kg, at least about 1.5 mg/kg, at least about 1.6 mg/kg, at least about 1.7 mg/kg, at least about 1.8 mg/kg, at least about 1.9 mg/kg, or at least about 2.0 mg/kg. In various embodiments, the dose of the SCN2A inhibitor or enhancer administered to a subject is at least about 5 mg/kg, at least about 10 mg/kg, at least about 15 mg/kg, at least about 20 mg/kg, at least about 25 mg/kg, at least about 30 mg/kg, at least about 35 mg/kg, at least about 40 mg/kg, at least about 45 mg/kg, at least about 50 mg/kg, at least about 55 mg/kg, at least about 60 mg/kg, at least about 65 mg/kg, at least about 70 mg/kg, at least about 75 mg/kg, at least about 80 mg/kg, at least about 85 mg/kg, at least about 90 mg/kg, at least about 95 mg/kg, or at least about 100 mg/kg.

In various embodiments, the dose of the SCN2A inhibitor or enhancer, such as a SCN2A nucleic acid inhibitor (e.g., ASO), administered to a subject can range between about 0.1 mg/kg and about 100 mg/kg. In various embodiments, the dose of the SCN2A inhibitor or enhancer administered to a subject can range between about 0.1 mg/kg and about 50 mg/kg. In various embodiments, the dose of the SCN2A inhibitor or enhancer administered to a subject can range between about 0.1 mg/kg and about 20 mg/kg. In various embodiments, the dose of the SCN2A inhibitor or enhancer administered to a subject can range between about 0.1 mg/kg and about 10 mg/kg. In various embodiments, the dose of the SCN2A inhibitor or enhancer administered to a subject can range between about 0.1 mg/kg and about 5 mg/kg. In various embodiments, the dose of the SCN2A inhibitor or enhancer administered to a subject can range between about 0.1 mg/kg and about 2 mg/kg. In various embodiments, the dose of the SCN2A inhibitor or enhancer administered to a subject can range between about 0.1 mg/kg and about 1.0 mg/kg. In various embodiments, the dose of the SCN2A inhibitor or enhancer administered to a subject can range between about 1 mg/kg and about 100 mg/kg. In various embodiments, the dose of the SCN2A inhibitor or enhancer administered to a subject can range between about 5 mg/kg and about 100 mg/kg. In various embodiments, the dose of the SCN2A inhibitor or enhancer administered to a subject can range between about 10 mg/kg and about 100 mg/kg. In various embodiments, the dose of the SCN2A inhibitor or enhancer administered to a subject can range between about 20 mg/kg and about 100 mg/kg. In various embodiments, the dose of the SCN2A inhibitor or enhancer administered to a subject can range between about 50 mg/kg and about 100 mg/kg. In various embodiments, the dose of the SCN2A inhibitor or enhancer administered to a subject can range between about 75 mg/kg and about 100 mg/kg. In various embodiments, the dose of the SCN2A inhibitor or enhancer administered to a subject can range between about 50 mg/kg and about 90 mg/kg. In various embodiments, the dose of the SCN2A inhibitor or enhancer administered to a subject can range between about 60 mg/kg and about 85 mg/kg. In various embodiments, the dose of the SCN2A inhibitor or enhancer administered to a subject can range between about 70 mg/kg and about 80 mg/kg. In various embodiments, the dose of the SCN2A inhibitor or enhancer administered to a subject can range between about 70 mg/kg and about 75 mg/kg. In various embodiments, the dose of the SCN2A inhibitor or enhancer administered to a subject can range between about 75 mg/kg and about 80 mg/kg.

In various embodiments, the SCN2A inhibitor or enhancer, such as a SCN2A nucleic acid inhibitor (e.g., ASO) is administered to a subject at or around the median effective dose (ED₅₀) (e.g., ±10% of ED₅₀ or ±20% of ED₅₀). In various embodiments, the SCN2A inhibitor or enhancer is administered to a subject at a high dose (e.g., an overdose), but limitedly or at a dose that does not negatively impact the subject (e.g., limited or no toxicity or lethality). In various embodiments, a high dose of the SCN2A inhibitor or enhancer is at least 50 times the ED₅₀ dose. In various embodiments, a high dose of the SCN2A inhibitor or enhancer is at least 100 times the ED₅₀ dose. In various embodiments, a high dose of the SCN2A inhibitor or enhancer is at least 200 times the ED₅₀ dose. As one example, the ED₅₀ dose can be about 0.5 mg/kg. A corresponding high dose of the SCN2A inhibitor or enhancer would therefore be, for example, at least about 25 mg/kg (i.e., at least about 50 times the ED₅₀ dose), at least about 50 mg/kg (i.e., at least about 100 times the ED₅₀ dose), or at least about 100 mg/kg (i.e., at least about 200 times the ED₅₀ dose).

In some embodiments, the SCN2A inhibitor or enhancer, such as a SCN2A nucleic acid inhibitor (e.g., ASO), is administered to a subject in an amount to decrease a SCN2A polypeptide to a level that is reduced by at least about 5%, such as at least about any of 10%, 15%, 20%, 25%, 30%, 35%, 40%, 45%, 50%, 55%, 60%, 65%, 70%, 75%, 80%, 85%, 90%, 95%, 96%, 97%, 98%, 99%, or 100%, as compared to a level of the SCN2A polypeptide prior to administration of the SCN2A inhibitor or enhancer or of the SCN2A polypeptide in a reference, such as a reference population. In certain aspects, the SCN2A inhibitors or enhancers described herein have a transitory effect, and one will readily appreciate that in some embodiments the reduced level and/or the level in a reference may reflect one or more measurements. For example, in some embodiments, the SCN2A inhibitor or enhancer is administered to a subject in an amount to decrease, for at least one measurable instance, a SCN2A polypeptide to a level that is reduced by at least about 5%, such as at least about any of 10%, 15%, 20%, 25%, 30%, 35%, 40%, 45%, 50%, 55%, 60%, 65%, 70%, 75%, 80%, 85%, 90%, 95%, 96%, 97%, 98%, 99%, or 100%, as compared to a level of the SCN2A polypeptide prior to administration of the SCN2A inhibitor or enhancer or of the SCN2A polypeptide in a reference, such as a reference population. In some embodiments, the reference population is a population of subjects not having the SCN2A-related disorder. In some embodiments, the method comprises a long-term administration strategy, such as two or more administrations of an SCN2A inhibitor or enhancer described herein. In some embodiments, the administration strategy comprises administering an SCN2A inhibitor or enhancer described herein at a desired frequency, wherein the frequency of administration is based on obtaining a decrease in one or more symptoms of a SCN2A-related disorder. In some embodiments, the administration strategy comprises administering an SCN2A inhibitor or enhancer described herein at a desired frequency, wherein the frequency of administration is based on obtaining a decrease or an increase in a level of a SCN2A polypeptide. In some embodiments, the administration strategy comprises administering an SCN2A inhibitor or enhancer described herein at a desired frequency, wherein the frequency of administration is based on tolerance, e.g., an acceptable level of toxicity. In some embodiments, the frequency of administration is daily, weekly, bi-weekly, monthly, quarterly, bi-annually, or yearly. In some embodiments, the frequency is adjusted based on efficacy of the SCN2A inhibitor or enhancer described herein.

Administration of SCN2A Inhibitors or SCN2A Enhancers and/or Pharmaceutical Compositions

The delivery of a SCN2A inhibitor or enhancer, such as a SCN2A nucleic acid inhibitor (e.g., ASO), to a cell e.g., a cell within a subject, such as a human subject e.g., a subject in need thereof, such as a subject having a SCN2A-related disorder, can be achieved in a number of different ways. For example, delivery may be performed by contacting a cell with a SCN2A inhibitor or enhancer either in vitro or in vivo. In vivo delivery may also be performed directly by administering a composition comprising a SCN2A inhibitor or enhancer to a subject. These alternatives are discussed further below.

In general, any method of delivering an inhibitor or enhancer molecule (in vitro or in vivo) can be adapted for use with a SCN2A inhibitor or enhancer as disclosed herein (see e.g., Akhtar S. and Julian R L., (1992) Trends Cell. Biol. 2(5):139-144 and WO 94/02595, which are incorporated herein by reference in their entireties). For in vivo delivery, factors to consider in order to deliver an inhibitor or enhancer molecule include, for example, biological stability of the delivered molecule, prevention of non-specific effects, and accumulation of the delivered molecule in the target tissue. The non-specific effects of an inhibitor or enhancer can be minimized by local administration, for example, by direct injection or implantation into a tissue or topically administering the preparation. Local administration to a treatment site maximizes local concentration of the agent, limits the exposure of the agent to systemic tissues that can otherwise be harmed by the agent or that can degrade the agent, and permits a lower total dose of the inhibitor or enhancer molecule to be administered.

In various embodiments, a disclosed SCN2A inhibitor or enhancer and any pharmaceutical composition thereof may be administered by one or several routes, including topically, intrathecally, intracisternally, parenterally, orally, rectally, buccally, sublingually, vaginally, pulmonarily, intratracheally, intranasally, transdermally, or intraduodenally. The term parenteral as used herein includes subcutaneous injections, intrapancreatic administration, intravenous, intracisternal, intrathecal, intramuscular, intraperitoneal, intrasternal injection or infusion techniques. For example, a SCN2A inhibitor or enhancer may be administered subcutaneously to a subject. In another example, a SCN2A inhibitor or enhancer may be administered orally to a subject. In some embodiments, the SCN2A inhibitor or enhancer, such as a SCN2A ASO, is administered intrathecally to a subject in a single infusion.

The SCN2A inhibitor or enhancer can be administered via any route of administration depending at least in part upon the particular condition to be treated. In various embodiments, the SCN2A inhibitor or enhancer is useful for the treatment of neurological conditions associated with SCN2A mutations, and therefore, the SCN2A inhibitor or enhancer is delivered to the central nervous system (CNS) and/or the peripheral nervous system (PNS). In such embodiments, the SCN2A inhibitor or enhancer is delivered to cells of the CNS or PNS, such as neuronal cells or cells of the spinal cord, for treating a SCN2A-related disorder, such as an epileptic encephalopathy. In various embodiments, the SCN2A inhibitor or enhancer can be delivered systemically with a delivery system enabling penetration of the blood brain barrier (e.g., exosomes or cell-penetrating peptide (CPP) conjugation). In various embodiments, the SCN2A inhibitor or enhancer can be delivered via inhalation (e.g. nasal inhalation, such as by formulating with CPP conjugated to glycol-polycaprolactone copolymers (Kanazawa, T., et al., “Delivery of siRNA to the brain using a combination of nose-to-brain delivery and cell-penetrating peptide-modified nano-micelles,” Biomaterials, 34 (2013), pp. 9220-9226). In various embodiments, the SCN2A inhibitor or enhancer is administered via local (direct) delivery to the CNS (e.g., brain or spinal column), such as by intrathecal, intramedullary, intracisternally, or intracerebroventricular injection.

Administration of an inhibitor or enhancer for treating SCN2A-related disorders can result in a decrease in the mortality rate of a population of subjects with a SCN2A-related disorder that are treated with an inhibitor or enhancer for treating SCN2A-related disorders, as compared to the mortality rate of a population of subjects with a SCN2A-related disorder that are not treated with an inhibitor or enhancer for treating SCN2A-related disorders. For example, the mortality rate may be decreased by more than 2% (e.g., more than 5%, 10%, or 25%). A decrease in the mortality rate of a population of treated subjects may be measured by any reproducible means, for example, by calculating for a population the average number of disease-related deaths per unit time following initiation of treatment with a compound or pharmaceutically acceptable salt of a compound described herein. A decrease in the mortality rate of a population may also be measured, for example, by calculating for a population the average number of disease-related deaths per unit time following completion of a first round of treatment with a compound or pharmaceutically acceptable salt of a compound described herein.

Administration of an inhibitor or enhancer for treating SCN2A-related disorders can result in an increase in average survival time of an individual subject or a population of subjects treated according to the methods disclosed herein with an inhibitor or enhancer for treating SCN2A-related disorders, as compared to an individual subject or a population of subjects who were not treated with an inhibitor or enhancer for treating SCN2A-related disorders. For example, the survival time of an individual subject or average survival time of population may be increased by more than 30 days (such as more than 60 days, 90 days, 120 days, 6 months, 1 year, 2 years, 5 years, or 10 years). An increase in survival time of an individual subject or in average survival time of a population may be measured by any reproducible means. An increase in survival time of an individual subject may be measured, for example, by calculating for an individual the length of survival time following the initiation of treatment with the compound described herein. An increase in average survival time of a population may be measured, for example, by calculating for the average length of survival time following initiation of treatment with the compound described herein. An increase in survival time of an individual subject may also be measured, for example, by calculating for an individual subject the length of survival time following completion of a first round of treatment with a compound or pharmaceutically acceptable salt of a compound described herein. An increase in average survival time of a population may also be measured, for example, by calculating for a population the average length of survival time following completion of a first round of treatment with a compound or pharmaceutically acceptable salt of a compound described herein.

### Timing of Administration

Embodiments disclosed herein involve administering an SCN2A inhibitor or enhancer, such as a SCN2A nucleic acid inhibitor (e.g., ASO), or a pharmaceutical composition comprising the SCN2A inhibitor or enhancer at a timepoint that is conducive for imparting therapeutic efficacy against a neurological disorder in a subject. Examples of neurological disorders include any of epilepsy, pediatric epilepsy, benign familial infantile seizures, early infantile epileptic encephalopathy, Ohtahara syndrome, infantile spasm syndrome (i.e., West Syndrome), generalized epilepsy with febrile seizures, migrating partial epilepsy of infancy, infantile spasms, and drug-resistant epilepsies. In some embodiments, the neurological disorder is early onset epileptic encephalopathy (EOEE), benign familial neonatal/infantile seizures (BFNIS), late seizure onset epileptic encephalopathy, or autism with or without epilepsy.

In various embodiments, the SCN2A inhibitor or enhancer, such as a SCN2A nucleic acid inhibitor (e.g., ASO), is administered to a subject after onset of a neurological disorder, such as after onset of a symptom thereof. For example, the SCN2A inhibitor may be administered after the subject experiences an early onset seizure. In various embodiments, the SCN2A inhibitor or enhancer is administered to a subject within 24 hours of the onset of a neurological disorder or a symptom caused by the neurological disorder (e.g., early onset seizure). In various embodiments, the SCN2A inhibitor or enhancer is administered to a subject within 2 days of the onset of a neurological disorder or a symptom caused by the neurological disorder (e.g., early onset seizure). In various embodiments, the SCN2A inhibitor or enhancer is administered to a subject within 3 days, within 4 days, within 5 days, within 6 days, or within 7 days of the onset of a neurological disorder or a symptom caused by the neurological disorder (e.g., early onset seizure). In various embodiments, the SCN2A inhibitor or enhancer is administered to a subject at within 1 week, within 2 weeks, within 3 weeks, within 4 weeks, within 5 weeks, within 6 weeks, within 7 weeks, within 8 weeks, within 9 weeks, or within 10 weeks of the onset of a neurological disorder or a symptom caused by the neurological disorder (e.g., early onset seizure). In various embodiments, the SCN2A inhibitor or enhancer is administered to a subject subsequent to 10 weeks after the onset of a neurological disorder or a symptom caused by the neurological disorder (e.g., early onset seizure).

### Subject for Administration of an SCN2A Inhibitor or Enhancer

Embodiments disclosed herein involve administering an SCN2A inhibitor or enhancer, such as a SCN2A nucleic acid inhibitor molecule (e.g., ASO), or a pharmaceutical composition comprising the SCN2A inhibitor or enhancer, to a subject for treating a neurological disorder in the subject.

In various embodiments, the subject is a mouse. In various embodiments, the subject is a non-human primate. In various embodiments, the subject is a human subject. In various embodiments, the subject is a pediatric subject (e.g., less than 18 years old). In various embodiments, the subject is an infant (e.g., less than 1 month old, less than 3 months old, less than 6 months old, less than 1 year old or less than 2 years old). In various embodiments, the subject is a subject in childhood (e.g., between ages 2-12 years old). In various embodiments, the subject is an adolescent subject (e.g., between ages 12-18 years old).

In particular embodiments, the subject is under the age of 18 years, under the age of 17 years, under the age of 16 years, under the age of 15 years, under the age of 14 years, under the age of 13 years, under the age of 12 years, under the age of 11 years, under the age of 10 years, under the age of 9 years, under the age of 8 years, under the age of 7 years, under the age of 6 years, under the age of 5 years, under the age of 4 years, under the age of 3 years, under the age of 2 years, under the age of 1 year old, under the age of 6 months, under the age of 3 months or under the age of 1 month.

In some embodiments, the subject is less than 2 years of age, and an SCN2A inhibitor or enhancer described herein, such as a SCN2A nucleic acid inhibitor molecule (e.g., ASO), is administered as a secondary indication. In some embodiments, the subject is between 2 and 12 years of age, and an SCN2A inhibitor or enhancer described herein, such as a SCN2A nucleic acid inhibitor molecule (e.g., ASO), is administered as an initial/primary indication. In some embodiments, the subject is between 13 and 17 years of age, and an SCN2A inhibitor or enhancer described herein, such as a SCN2A ASO, is administered as a secondary indication.

### Effects of SCN2A Inhibitor and SCN2A Enhancers

The therapeutic effects of administering SCN2A inhibitors or enhancers, such as a SCN2A nucleic acid inhibitor molecule (e.g., ASO), for reducing SCN2A activity or increasing SCN2A activity, respectively, can be evaluated according to one or more criteria. For example, criteria can include a change in a frequency of seizures following treatment, a change in survival time (e.g., mortality) following onset of disease or disorder, and/or a change in levels of SCN2A protein or SCN2A mRNA transcripts in the subject following treatment. A control subject or group of subjects may be used to assess the therapeutic effects of administering SCN2A inhibitors or enhancers. For example, a control subject or group of subjects may be treated with a control treatment that differs from the SCN2A inhibitors or enhancers described herein. In some embodiments, the effective amount of the SCN2A inhibitor or enhancer described herein, such as a SCN2A nucleic acid inhibitor molecule (e.g., ASO), reduces seizures (such as refractory seizures) by at least about 50%, such as at least about any of 55%, 60%, 65%, 70%, 75%, 80%, 85%, 90%, or 95%, compared to the baseline at minimum.

The level of SCN2A protein or SCN2A mRNA transcripts that are expressed in a subject may be determined using any method known in the art for assessing mRNA expression. In one embodiment, a sample is obtained from the subject, and the sample is assessed for levels of SCN2A mRNA transcripts. Examples of a sample obtained from the subject include amniotic fluid, aqueous humor, bile, lymph, breast milk, interstitial fluid, blood, blood plasma, cerumen (earwax), Cowper's fluid (pre-ejaculatory fluid), chyle, chyme, female ejaculate, menses, mucus, saliva, urine, vomit, tears, vaginal lubrication, sweat, serum, semen, sebum, pus, pleural fluid, cerebrospinal fluid, synovial fluid, intracellular fluid, and vitreous humour. In particular embodiments, the sample is a cerebrospinal fluid. Therefore, the levels of SCN2A mRNA transcripts and/or SCN2A protein in the cerebrospinal fluid may indicate the levels of SCN2A mRNA transcripts and/or SCN2A protein in the subject's central nervous system. In particular embodiments, the sample is a tissue sample obtained from the subject (e.g., a tissue biopsy). Examples of a tissue sample include tissue from a subject's central nervous system such as brain tissue (e.g., brain cortex) or spinal cord tissue. As such, a change in the level of SCN2A protein or SCN2A mRNA transcripts in the subject's brain tissue (e.g., brain cortex) and/or spinal cord tissue can be determined according to the methods described herein. In some embodiments, the sample is obtained from the subject prior to initiation of a method of treatment described herein. In some embodiments, the sample, including additional samples, are obtained from the subject following initiation of a method of treatment described herein.

SCN2A mRNA transcripts may be extracted from the sample using RNA extraction techniques including, for example, using acid phenol/guanidine isothiocyanate extraction (RNAzol B; Biogenesis), RNEASY™ RNA preparation kits (Qiagen) or PAXgene (PreAnalytix, Switzerland). Typical assay formats utilizing ribonucleic acid hybridization include nuclear run-on assays, RT-PCR, RNase protection assays, northern blotting, in situ hybridization, and microarray analysis. In some embodiments, the level of SCN2A mRNA transcripts is determined using a nucleic acid probe. The term “probe,” as used herein, refers to any molecule that is capable of selectively binding to a specific SCN2A sequence, e.g., to an mRNA or polypeptide. Probes can be synthesized by one of skill in the art or derived from appropriate biological preparations. Probes may be specifically designed to be labeled. Examples of molecules that can be utilized as probes include, but are not limited to, RNA, DNA, proteins, antibodies, and organic molecules. In various embodiments, SCN2A mRNA transcripts are detected using hybridization or amplification assays that include, but are not limited to, Southern or northern analyses, polymerase chain reaction (PCR) analyses, and probe arrays. One method for the determination of mRNA levels involves contacting the isolated mRNA with a nucleic acid molecule (probe) that can hybridize to SCN2A mRNA. In one embodiment, the mRNA is immobilized on a solid surface and contacted with a probe, for example by running the isolated mRNA on an agarose gel and transferring the mRNA from the gel to a membrane, such as nitrocellulose. In an alternative embodiment, the probe(s) are immobilized on a solid surface and the mRNA is contacted with the probe(s), for example, in an AFFYMETRIX gene chip array. A skilled artisan can readily adapt known mRNA detection methods for use in determining the level of SCN2A mRNA.

An alternative method for determining the expression level of SCN2A in a sample involves the process of nucleic acid amplification and/or reverse transcriptase (to prepare cDNA) of nucleic acids, for example mRNA, in the sample, e.g., by RT-PCR (the experimental embodiment set forth in Mullis, 1987, U.S. Pat. No. 4,683,202), ligase chain reaction (Barany (1991) Proc. Natl. Acad. Sci. USA 88:189-193), self-sustained sequence replication (Guatelli et al. (1990) Proc. Natl. Acad. Sci. USA 87:1874-1878), transcriptional amplification system (Kwoh et al. (1989) Proc. Natl. Acad. Sci. USA 86:1173-1177), Q-Beta Replicase (Lizardi et al. (1988) Bio/Technology 6:1197), rolling circle replication (Lizardi et al., U.S. Pat. No. 5,854,033) or any other nucleic acid amplification method, followed by the detection of the amplified molecules using techniques well known to those of skill in the art. These detection schemes are especially useful for the detection of nucleic acid molecules if such molecules are present in very low numbers. In particular aspects disclosed herein, the level of expression of SCN2A is determined by quantitative fluorogenic RT-PCR (i.e., the TAQMAN™ System) or the DUAL-GLO® Luciferase assay.

The level of SCN2A protein expression may be determined using any method known in the art for the measurement of protein levels. Such methods include, for example, electrophoresis, capillary electrophoresis, high performance liquid chromatography (HPLC), thin layer chromatography (TLC), hyperdiffusion chromatography, fluid or gel precipitin reactions, absorption spectroscopy, colorimetric assays, spectrophotometric assays, flow cytometry, immunodiffusion (single or double), immunoelectrophoresis, western blotting, radioimmunoassay (RIA), enzyme-linked immunosorbent assays (ELISAs), immunofluorescent assays, electrochemiluminescence assays, and the like. Such assays can also be used for the detection of proteins indicative of the presence or replication of SCN2A proteins.

### Example Methods

In certain aspects, the methods taught herein, and aspects thereof, are presented in a modular manner. Such disclosure is not intended to limit the scope of the description, and one will readily appreciate the combination of disclosed features for the methods of treating a subject taught herein. Certain example methods are presented below and are not intended to limit the scope of methods taught herein.

In some embodiments, provided is a method for treating a subject with a SCN2A-related disorder caused by a gain-of-function SCN2A mutation, the method comprising administering an effective amount of a SCN2A ASO to the subject. In some embodiments, the subject has experienced an early onset seizure and comprises a SCN2A protein comprising A263V, E430A, R1882Q, G879R, G1593R, K1502N, V1601L, G211D, S1780I, and/or D343H. In some embodiments, the subject is a pediatric subject. In some embodiments, the effective amount of the SCN2A ASO is between about 0.1 mg/kg and about 100 mg/kg. In some embodiments, the effective amount of the SCN2A ASO decreases a SCN2A polypeptide to a level that is reduced by at least about 5%, such as at least about any of 10%, 15%, 20%, 25%, 30%, 35%, 40%, 45%, 50%, 55%, 60%, 65%, 70%, 75%, 80%, 85%, 90%, 95%, 96%, 97%, 98%, 99%, or 100%, as compared to a level of the SCN2A polypeptide prior to administration of the SCN2A ASO or of the SCN2A polypeptide in a reference, such as a reference population. In some embodiments, the administration of the SCN2A ASO decreases the SCN2A polypeptide level in the central nervous system (CNS). In some embodiments, the SCN2A ASO is a gapmer. In some embodiments, the SCN2A ASO is administered to the subject after onset of the SCN2A-related disorder in the subject, such as within 3 days of the observation of a symptom of the SCN2A-related disorder caused by a gain-of-function SCN2A mutation. In some embodiments, the SCN2A ASO is administered intrathecally. In some embodiments, the method comprises administering the SCN2A ASO quarterly. In some embodiments, provided is a method for treating a subject with early onset epileptic encephalopathy (EOEE), the method comprising administering an effective amount of a SCN2A ASO to the subject. In some embodiments, the SCN2A ASO is administered to the subject after onset of EOEE in the subject, such as within 3 days of the observation of a symptom of EOEE.

In some embodiments, provided is a method for treating a subject with a SCN2A-related disorder caused by a loss-of-function SCN2A mutation, the method comprising administering an effective amount of a SCN2A enhancer ASO to the subject, wherein the SCN2A enhancer is an ASO. In some embodiments, the subject has not experienced an early onset seizure and comprises a SCN2A protein comprising M951R, F1375V, R853Q, A1773T, R571H, K1422E, D195G, Y428, K507E, Y1771H, and/or S1758R. In some embodiments, the SCN2A-related disorder is selected from the group consisting benign familial neonatal/infantile seizures (BFNIS), late seizure onset epileptic encephalopathy, and autism with or without epilepsy. In some embodiments, the subject is a pediatric subject. In some embodiments, the effective amount of the SCN2A ASO is between about 0.1 mg/kg and about 100 mg/kg. In some embodiments, the effective amount of the SCN2A ASO increases a SCN2A polypeptide to a level that is increased by at least about 5%, such as at least about any of 10%, 15%, 20%, 25%, 30%, 35%, 40%, 45%, 50%, 55%, 60%, 65%, 70%, 75%, 80%, 85%, 90%, 95%, 96%, 97%, 98%, 99%, or 100%, as compared to a level of the SCN2A polypeptide prior to administration of the SCN2A ASO or of the SCN2A polypeptide in a reference, such as a reference population. In some embodiments, the administration of the SCN2A ASO increases the SCN2A polypeptide level in the CNS. In some embodiments, the SCN2A ASO is a gapmer. In some embodiments, the SCN2A ASO is administered to the subject after onset of the SCN2A-related disorder in the subject, such as within 3 days of the observation of a symptom of the SCN2A-related disorder caused by a loss-of-function SCN2A mutation. In some embodiments, the SCN2A ASO is administered intrathecally. In some embodiments, the method comprises administering the SCN2A ASO quarterly. In some embodiments, the SCN2A-related disorder caused by a loss-of-function SCN2A mutation is benign familial neonatal/infantile seizures (BFNIS), late seizure onset epileptic encephalopathy, autism with epilepsy.

### EXAMPLES

### Example 1. Analysis of Patients with Mutant SCN2A Neurological Disorders to Identify Markers that Correlate with Gain-of-Function Mutations

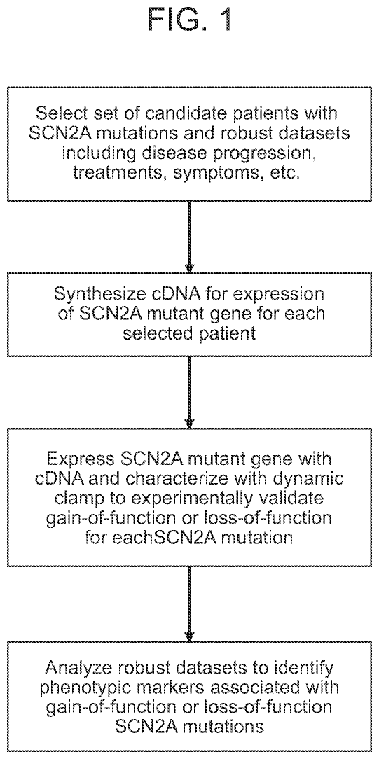

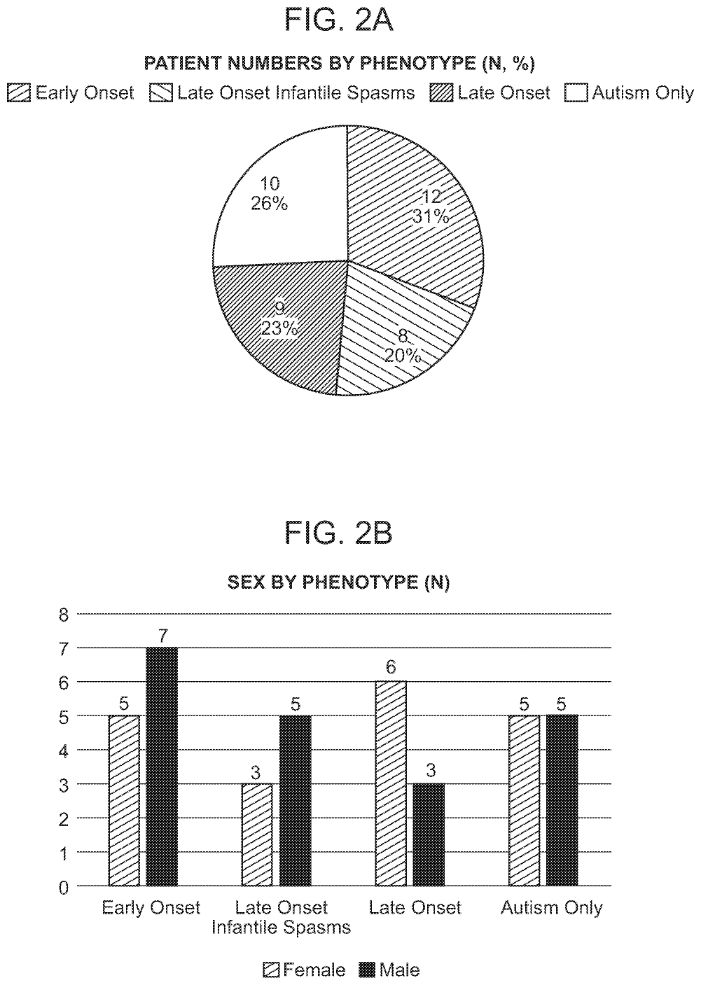

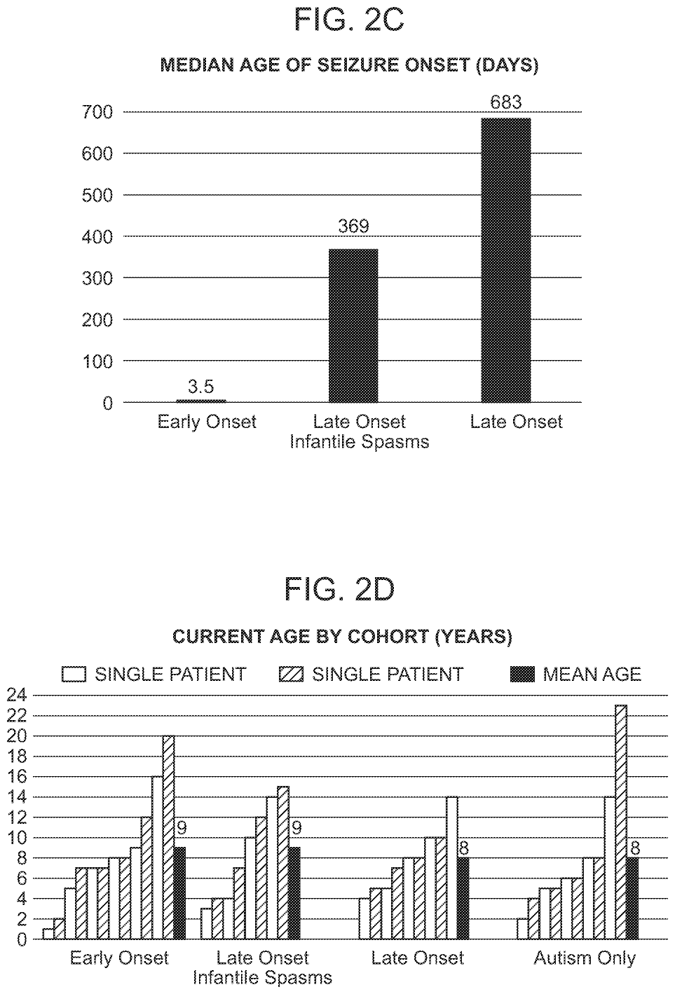

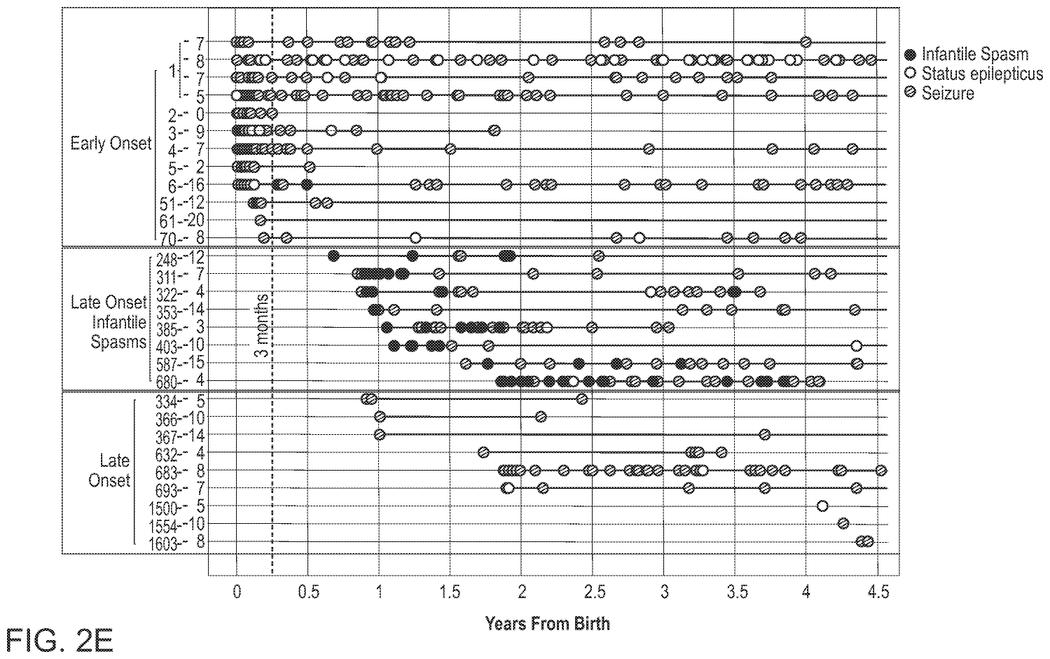

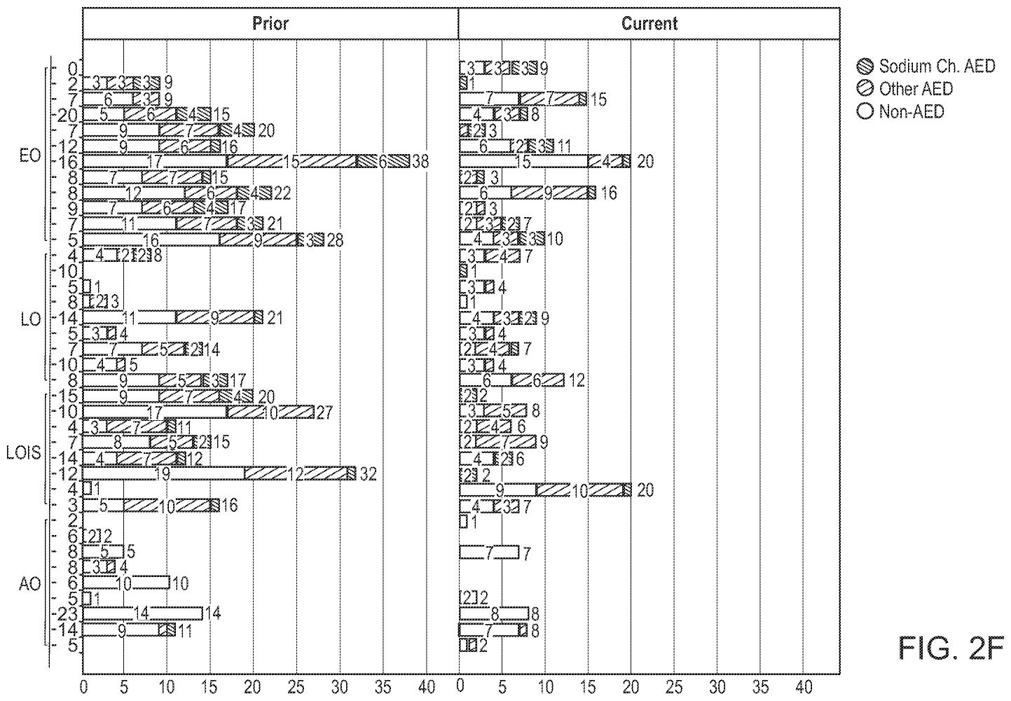

The goal of the study was to better understand the SCN2A patient population so as to provide the best and most appropriate therapies possible, particularly in light of the advent of new therapies under development that inhibit SCN2A and are therefore of particular use in treatment of gain-of-function SCN2A mutations and associated neurological disorders. FIG. 1 highlights the overall flowchart of the study. In the first step, a set of 39 patients with SCN2A mutation neurological disorders were selected. Characteristics of the patients are shown in FIGS. 2A-F, including the percentage of patients with each phenotype studied (FIG. 2A), their sex (FIG. 2B), median age of seizure onset (FIG. 2C), age at the time of study (FIG. 2D), seizure history from birth to age 4.5 (FIG. 2E), and prior medication counts and medication counts at the time of study (FIG. 2F).

Per FIG. 1, the next step was to synthesize cDNAs expressing the SCN2A mutations. Missense mutations were selected for the first portion of this analysis. The cDNAs were used to transiently express the SCN2A mutations in CHO for dynamic clamp experiments to experimentally determine the if the SCN2A mutation was a gain-of-function mutation or a loss-of-function mutation.

For the final step, rigorous analysis was applied to the robust datasets searching for markers or sets of markers that correlated reasonably well with gain-of-function mutations or loss-of-function mutations so that the marker or sets of markers could be used in lieu of experimental verification of the SCN2A mutation as a gain-of-function mutation or loss-of-function, so that treatment with an SCN2A inhibitor or enhancer, respectively, could be initiated as soon as the marker or sets of markers had been assessed for a given patient with an SCN2A mutation neurological disorder. This robust analysis included a detailed analysis of, among other markers, electroencephalogram data included in the robust datasets.

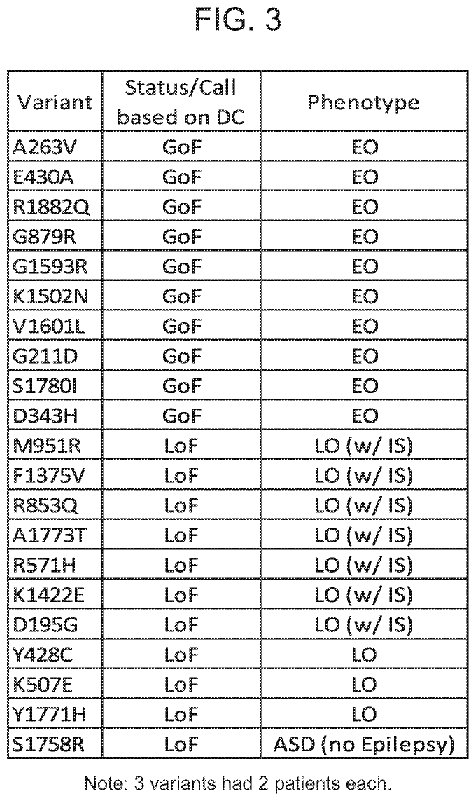

Before the rigorous analysis was initiated, the SCN2A mutations were grouped by the following gross phenotypes: (i) early onset seizures, (ii) late onset seizures with infantile spasms, (iii) late onset without infantile spasms, and (iv) autism spectrum disorder without seizures. The groupings are shown in FIG. 3 with the specific SCN2A variant and the nature of the mutation as gain-of-function (GoF) or loss-of-function (LoF). As shown in FIG. 3, surprisingly, all of the patients that had their first seizure in their first three months of life have gain-of-function SCN2A mutations. Further, all of the patients that had their first seizure after their first three months of life, or do not have seizures, unexpectedly have loss-of-function SCN2A mutations.

### Example 2. Disease Impact and Burden in Patients with SCN2A-Related Developmental and Epileptic Encephalopathy

SCN2A-related developmental and epileptic encephalopathy (DEE) comprises a subset of rare neurodevelopmental genetic disorders associated with a broad epilepsy phenotype, developmental impairments, intellectual disability, and autism. As a rare condition with significant heterogeneity, comprehensive understanding of disease burden and progression is limited. The goal of this study was to explore the burden and impact of SCN2A-DEE on patients and caregivers using clinical data generated via a patient-consented, real-world data platform.

Real-world clinical data were extracted from patients with an SCN2A variant using an approach combining machine learning and expert human review to streamline generation of research-grade data from unstructured health records spanning about 10 years. Extracted data, including seizure history, medication use, comorbidities, and developmental milestones, were classified by phenotype based on age at seizure onset, type of seizure at initial presentation, variant type, and variant functional characterization using dynamic action potential clamp analysis. SCN2A variants producing increased or decreased action potential relative to wild type were classified as probable gain-of-function or loss-of-function, respectively.

Of 49 enrolled patients, 45 had data available for analysis. Four emergent phenotypes (Table 1) were identified across three groups: early onset epilepsy (EO, 33.5%), late onset epilepsy (LO, 42%), and autism without epilepsy (AO, 24.5%). The LO group contained two distinct phenotypes based on age and type of seizure at onset: the first, characterized by initial seizure presentation with infantile spasms (LOIS, 20%); the second characterized by seizures other than infantile spasms and presenting later in life (LO, 22%). The AO group was characterized by developmental delay and autism without epilepsy.

Mean patient age across cohorts was 8 years (range 0.8-23.3), with similar percentages of male and female patients. Mean age at seizure onset was 5.1 (1-44) days for those classified as EO. Fifty-three percent of patients with seizures experienced status epilepticus at least once. Mean number of medications prescribed over the lifetime of each patient was 18.1 (0-58). Patients had multiple comorbidities, with global developmental delay reported in almost all patients (97%), and hypotonia, GERD, autism, and sleep disorders reported in >50%. Greater than 60% of patients had not achieved age-appropriate physical developmental milestones.

TABLE 1
SCN2A Phenotypic Classification based on Clinical Features and Biophysical Characterization

| | | Late Onset | | Autism without |
|---|---|---|---|---|
| | Early Onset | Late Onset with | | Epilepsy |
| | Early Onset | Infantile Spasms | Late Onset | Autism Only |
| | (EO) | (LOIS) | (LO) | (AO) |
| Functional | Gain of function | Loss of function | Loss of function | Loss of function |
| characterization | | | | |
| of genetic variant | | | | |
| Mutation type | Missense | Missense | Nonsense | Nonsense |
| | | | Frameshift | Frameshift |
| | | | Missense | Missense |
| Age of seizure | Typically presents | Typically presents | Typically presents | n/a |
| onset | in the first few | between 6 to | between 18 months | |
| | days of life, up to | 12 months of life | to 4 years of life | |
| | 3 months of life | | | |
| First seizure type | Seizures other | Infantile spasms | Seizures other than | n/a |
| | than infantile | always first seizure | infantile spasms | |
| | spasms | presentation | | |
| Additional | — | — | Autism typically | Developmental |
| phenotypic | | | presents prior to | delay and |
| characteristics | | | seizures | autism |

Using a novel real-world data platform and functional variant characterization, this study provides unprecedented insight into clinical phenotypes, disease burden and treatment patterns in SCN2A. The results of the study demonstrate that symptoms are diverse and extend beyond epilepsy, with patient burden compounded by comorbidities, treatment use, and procedural interventions. The findings of the study aid development of more effective and targeted therapies for better outcomes in SCN2A.

### SEQUENCES

SEQUENCES

Homo sapiens SCN2A protein (SEQ ID NO: 1):
MAQSVLVPPGPDSFRFFTRESLAAIEQRIAEEKAKRPKQERKDEDDENGPKPNSDLEA
GKSLPFIYGDIPPEMVSVPLEDLDPYYINKKTFIVLNKGKAISRFSATPALYILTPFNPIR
KLAIKILVHSLFNMLIMCTILTNCVFMTMSNPPDWTKNVEYTFTGIYTFESLIKILARG
FCLEDFTFLRDPWNWLDFTVITFAYVTEFVDLGNVSALRTFRVLRALKTISVIPGLKTI
VGALIQSVKKLSDVMILTVFCLSVFALIGLQLFMGNLRNKCLQWPPDNSSFEINITSFF
NNSLDGNGTTFNRTVSIFNWDEYIEDKSHFYFLEGQNDALLCGNSSDAGQCPEGYIC
VKAGRNPNYGYTSFDTFSWAFLSLFRLMTQDFWENLYQLTLRAAGKTYMIFFVLVIF
LGSFYLINLILAVVAMAYEEQNQATLEEAEQKEAEFQQMLEQLKKQQEEAQAAAAA
ASAESRDFSGAGGIGVFSESSSVASKLSSKSEKELKNRRKKKKQKEQSGEEEKNDRV
RKSESEDSIRRKGFRFSLEGSRLTYEKRFSSPHQSLLSIRGSLFSPRRNSRASLFSFRGR
AKDIGSENDFADDEHSTFEDNDSRRDSLFVPHRHGERRHSNVSQASRASRVLPILPM
NGKMHSAVDCNGVVSLVGGPSTLTSAGQLLPEGTTTETEIRKRRSSSYHVSMDLLED
PTSRQRAMSIASILTNTMEELEESRQKCPPCWYKFANMCLIWDCCKPWLKVKHLVN
LVVMDPFVDLAITICIVLNTLFMAMEHYPMTEQFSSVLSVGNLVFTGIFTAEMFLKIIA
MDPYYYFQEGWNIFDGFIVSLSLMELGLANVEGLSVLRSFRLLRVFKLAKSWPTLN
MLIKIIGNSVGALGNLTLVLAIIVFIFAVVGMQLFGKSYKECVCKISNDCELPRWHMH
DFFHSFLIVFRVLCGEWIETMWDCMEVAGQTMCLTVFMMVMVIGNLVVLNLFLAL
LLSSFSSDNLAATDDDNEMNNLQIAVGRMQKGIDFVKRKIREFIQKAFVRKQKALDE
IKPLEDLNNKKDSCISNHTTIEIGKDLNYLKDGNGTTSGIGSSVEKYVVDESDYMSFIN
NPSLTVTVPIAVGESDFENLNTEEFSSESDMEESKEKLNATSSSEGSTVDIGAPAEGEQ
PEVEPEESLEPEACFTEDCVRKFKCCQISIEEGKGKLWWNLRKTCYKIVEHNWFETFI
VFMILLSSGALAFEDIYIEQRKTIKTMLEYADKVFTYIFILEMLLKWVAYGFQVYFTN
AWCWLDFLIVDVSLVSLTANALGYSELGAIKSLRTLRALRPLRALSRFEGMRVVVN
ALLGAIPSIMNVLLVCLIFWLIFSIMGVNLFAGKFYHCINYTTGEMFDVSVVNNYSEC
KALIESNQTARWKNVKVNFDNVGLGYLSLLQVATFKGWMDIMYAAVDSRNVELQP
KYEDNLYMYLYFVIFIIFGSFFTLNLFIGVIIDNFNQQKKKFGGQDIFMTEEQKKYYNA
MKKLGSKKPQKPIPRPANKFQGMVFDFVTKQVFDISIMILICLNMVTMMVETDDQSQ
EMTNILYWINLVFIVLFTGECVLKLISLRYYYFTIGWNIFDFVVVILSIVGMFLAELIEK
YFVSPTLFRVIRLARIGRILRLIKGAKGIRTLLFALMMSLPALFNIGLLLFLVMFIYAIF
GMSNFAYVKREVGIDDMFNFETFGNSMICLFQITTSAGWDGLLAPILNSGPPDCDPD
KDHPGSSVKGDCGNPSVGIFFFVSYIIISFLVVVNMYIAVILENFSVATEESAEPLSEDD
FEMFYEVWEKFDPDATQFIEFAKLSDFADALDPPLLIAKPNKVQLIAMDLPMVSGDR
IHCLDILFAFTKRVLGESGEMDALRIQMEERFMASNPSKVSYEPITTTLKRKQEEVSAI
IIQRAYRRYLLKQKVKKVSSIYKKDKGKECDGTPIKEDTLIDKLNENSTPEKTDMTPS
TTSPPSYDSVTKPEKEKFEKDKSEKEDKGKDIRESKK
Homo sapiens SCN2A RNA (SEQ ID NO: 2):
AAGCATGATGGAATTTTAGCTGCAGTCTTCTTGGTGCCAGCTTATCAATCCCAAA
CTCTGGGTGTAAAAGATTCTACAGGGCACTTTCTTATGCAAGGAGCTAAACAGTG
ATTAAAGGAGCAGGATGAAAAGATGGCACAGTCAGTGCTGGTACCGCCAGGACC
TGACAGCTTCCGCTTCTTTACCAGGGAATCCCTTGCTGCTATTGAACAACGCATT
GCAGAAGAGAAAGCTAAGAGACCCAAACAGGAACGCAAGGATGAGGATGATGA
AAATGGCCCAAAGCCAAACAGTGACTTGGAAGCAGGAAAATCTCTTCCATTTAT
TTATGGAGACATTCCTCCAGAGATGGTGTCAGTGCCCCTGGAGGATCTGGACCCC
TACTATATCAATAAGAAAACGTTTATAGTATTGAATAAAGGGAAAGCAATCTCTC
GATTCAGTGCCACCCCTGCCCTTTACATTTTAACTCCCTTCAACCCTATTAGAAAA
TTAGCTATTAAGATTTTGGTACATTCTTTATTCAATATGCTCATTATGTGCACGAT
TCTTACCAACTGTGTATTTATGACCATGAGTAACCCTCCAGACTGGACAAAGAAT
GTGGAGTATACCTTTACAGGAATTTATACTTTTGAATCACTTATTAAAATACTTGC
AAGGGGCTTTTGTTTAGAAGATTTCACATTTTTACGGGATCCATGGAATTGGTTG
GATTTCACAGTCATTACTTTTGCATATGTGACAGAGTTTGTGGACCTGGGCAATG
TCTCAGCGTTGAGAACATTCAGAGTTCTCCGAGCATTGAAAACAATTTCAGTCAT
TCCAGGCCTGAAGACCATTGTGGGGGCCCTGATCCAGTCAGTGAAGAAGCTTTCT
GATGTCATGATCTTGACTGTGTTCTGTCTAAGCGTGTTTGCGCTAATAGGATTGCA
GTTGTTCATGGGCAACCTACGAAATAAATGTTTGCAATGGCCTCCAGATAATTCT
TCCTTTGAAATAAATATCACTTCCTTCTTTAACAATTCATTGGATGGGAATGGTAC
TACTTTCAATAGGACAGTGAGCATATTTAACTGGGATGAATATATTGAGGATAAA
AGTCACTTTTATTTTTTAGAGGGGCAAAATGATGCTCTGCTTTGTGGCAACAGCT
CAGATGCAGGCCAGTGTCCTGAAGGATACATCTGTGTGAAGGCTGGTAGAAACC
CCAACTATGGCTACACGAGCTTTGACACCTTTAGTTGGGCCTTTTTGTCCTTATTT
CGTCTCATGACTCAAGACTTCTGGGAAAACCTTTATCAACTGACACTACGTGCTG
CTGGGAAAACGTACATGATATTTTTTGTGCTGGTCATTTTCTTGGGCTCATTCTAT
CTAATAAATTTGATCTTGGCTGTGGTGGCCATGGCCTATGAGGAACAGAATCAGG
CCACATTGGAAGAGGCTGAACAGAAGGAAGCTGAATTTCAGCAGATGCTCGAAC
AGTTGAAAAAGCAACAAGAAGAAGCTCAGGCGGCAGCTGCAGCCGCATCTGCTG
AATCAAGAGACTTCAGTGGTGCTGGTGGGATAGGAGTTTTTTCAGAGAGTTCTTC
AGTAGCATCTAAGTTGAGCTCCAAAAGTGAAAAAGAGCTGAAAAACAGAAGAA
AGAAAAAGAAACAGAAAGAACAGTCTGGAGAAGAAGAGAAAAATGACAGAGTC
CGAAAATCGGAATCTGAAGACAGCATAAGAAGAAAAGGTTTCCGTTTTTCCTTG
GAAGGAAGTAGGCTGACATATGAAAAGAGATTTTCTTCTCCACACCAGTCCTTAC
TGAGCATCCGTGGCTCCCTTTTCTCTCCAAGACGCAACAGTAGGGCGAGCCTTTT
CAGCTTCAGAGGTCGAGCAAAGGACATTGGCTCTGAGAATGACTTTGCTGATGA
TGAGCACAGCACCTTTGAGGACAATGACAGCCGAAGAGACTCTCTGTTCGTGCC
GCACAGACATGGAGAACGGCGCCACAGCAATGTCAGCCAGGCCAGCCGTGCCTC
CAGGGTGCTCCCCATCCTGCCCATGAATGGGAAGATGCATAGCGCTGTGGACTG
CAATGGTGTGGTCTCCCTGGTCGGGGGCCCTTCTACCCTCACATCTGCTGGGCAG
CTCCTACCAGAGGGCACAACTACTGAAACAGAAATAAGAAAGAGACGGTCCAGT
TCTTATCATGTTTCCATGGATTTATTGGAAGATCCTACATCAAGGCAAAGAGCAA
TGAGTATAGCCAGTATTTTGACCAACACCATGGAAGAACTTGAAGAATCCAGAC
AGAAATGCCCACCATGCTGGTATAAATTTGCTAATATGTGTTTGATTTGGGACTG
TTGTAAACCATGGTTAAAGGTGAAACACCTTGTCAACCTGGTTGTAATGGACCCA
TTTGTTGACCTGGCCATCACCATCTGCATTGTCTTAAATACACTCTTCATGGCTAT
GGAGCACTATCCCATGACGGAGCAGTTCAGCAGTGTACTGTCTGTTGGAAACCTG
GTCTTCACAGGGATCTTCACAGCAGAAATGTTTCTCAAGATAATTGCCATGGATC
CATATTATTACTTTCAAGAAGGCTGGAATATTTTTGATGGTTTTATTGTGAGCCTT
AGTTTAATGGAACTTGGTTTGGCAAATGTGGAAGGATTGTCAGTTCTCCGATCAT
TCCGGCTGCTCCGAGTTTTCAAGTTGGCAAAATCTTGGCCAACTCTAAATATGCT
AATTAAGATCATTGGCAATTCTGTGGGGGCTCTAGGAAACCTCACCTTGGTATTG
GCCATCATCGTCTTCATTTTTGCTGTGGTCGGCATGCAGCTCTTTGGTAAGAGCTA
CAAAGAATGTGTCTGCAAGATTTCCAATGATTGTGAACTCCCACGCTGGCACATG
CATGACTTTTTCCACTCCTTCCTGATCGTGTTCCGCGTGCTGTGTGGAGAGTGGAT
AGAGACCATGTGGGACTGTATGGAGGTCGCTGGCCAAACCATGTGCCTTACTGTC
TTCATGATGGTCATGGTGATTGGAAATCTAGTGGTTCTGAACCTCTTCTTGGCCTT
GCTTTTGAGTTCCTTCAGTTCTGACAATCTTGCTGCCACTGATGATGATAACGAA
ATGAATAATCTCCAGATTGCTGTGGGAAGGATGCAGAAAGGAATCGATTTTGTTA
AAAGAAAAATACGTGAATTTATTCAGAAAGCCTTTGTTAGGAAGCAGAAAGCTT
TAGATGAAATTAAACCGCTTGAAGATCTAAATAATAAAAAAGACAGCTGTATTT
CCAACCATACCACCATAGAAATAGGCAAAGACCTCAATTATCTCAAAGACGGAA
ATGGAACTACTAGTGGCATAGGCAGCAGTGTAGAAAAATATGTCGTGGATGAAA
GTGATTACATGTCATTTATAAACAACCCTAGCCTCACTGTGACAGTACCAATTGC
TGTTGGAGAATCTGACTTTGAAAATTTAAATACTGAAGAATTCAGCAGCGAGTCA
GATATGGAGGAAAGCAAAGAGAAGCTAAATGCAACTAGTTCATCTGAAGGCAGC
ACGGTTGATATTGGAGCTCCCGCCGAGGGAGAACAGCCTGAGGTTGAACCTGAG
GAATCCCTTGAACCTGAAGCCTGTTTTACAGAAGACTGTGTACGGAAGTTCAAGT
GTTGTCAGATAAGCATAGAAGAAGGCAAAGGGAAACTCTGGTGGAATTTGAGGA
AAACATGCTATAAGATAGTGGAGCACAATTGGTTCGAAACCTTCATTGTCTTCAT
GATTCTGCTGAGCAGTGGGGCTCTGGCCTTTGAAGATATATACATTGAGCAGCGA
AAAACCATTAAGACCATGTTAGAATATGCTGACAAGGTTTTCACTTACATATTCA
TTCTGGAAATGCTGCTAAAGTGGGTTGCATATGGTTTTCAAGTGTATTTTACCAAT
GCCTGGTGCTGGCTAGACTTCCTGATTGTTGATGTCTCACTGGTTAGCTTAACTGC
AAATGCCTTGGGTTACTCAGAACTTGGTGCCATCAAATCCCTCAGAACACTAAGA
GCTCTGAGGCCACTGAGAGCTTTGTCCCGGTTTGAAGGAATGAGGGTTGTTGTAA
ATGCTCTTTTAGGAGCCATTCCATCTATCATGAATGTACTTCTGGTTTGTCTGATC
TTTTGGCTAATATTCAGTATCATGGGAGTGAATCTCTTTGCTGGCAAGTTTTACCA
TTGTATTAATTACACCACTGGAGAGATGTTTGATGTAAGCGTGGTCAACAACTAC
AGTGAGTGCAAAGCTCTCATTGAGAGCAATCAAACTGCCAGGTGGAAAAATGTG
AAAGTAAACTTTGATAACGTAGGACTTGGATATCTGTCTCTACTTCAAGTAGCCA
CGTTTAAGGGATGGATGGATATTATGTATGCAGCTGTTGATTCACGAAATGTAGA
ATTACAACCCAAGTATGAAGACAACCTGTACATGTATCTTTATTTTGTCATCTTTA
TTATTTTTGGTTCATTCTTTACCTTGAATCTTTTCATTGGTGTCATCATAGATAACT
TCAACCAACAGAAAAAGAAGTTTGGAGGTCAAGACATTTTTATGACAGAAGAAC
AGAAGAAATACTACAATGCAATGAAAAAACTGGGTTCAAAGAAACCACAAAAA
CCCATACCTCGACCTGCTAACAAATTCCAAGGAATGGTCTTTGATTTTGTAACCA
AACAAGTCTTTGATATCAGCATCATGATCCTCATCTGCCTTAACATGGTCACCAT
GATGGTGGAAACCGATGACCAGAGTCAAGAAATGACAAACATTCTGTACTGGAT
TAATCTGGTGTTTATTGTTCTGTTCACTGGAGAATGTGTGCTGAAACTGATCTCTC
TTCGTTACTACTATTTCACTATTGGATGGAATATTTTTGATTTTGTGGTGGTCATT
CTCTCCATTGTAGGAATGTTTCTGGCTGAACTGATAGAAAAGTATTTTGTGTCCCC
TACCCTGTTCCGAGTGATCCGTCTTGCCAGGATTGGCCGAATCCTACGTCTGATC
AAAGGAGCAAAGGGGATCCGCACGCTGCTCTTTGCTTTGATGATGTCCCTTCCTG
CGTTGTTTAACATCGGCCTCCTTCTTTTCCTGGTCATGTTCATCTACGCCATCTTTG
GGATGTCCAATTTTGCCTATGTTAAGAGGGAAGTTGGGATCGATGACATGTTCAA
CTTTGAGACCTTTGGCAACAGCATGATCTGCCTGTTCCAAATTACAACCTCTGCT
GGCTGGGATGGATTGCTAGCACCTATTCTTAATAGTGGACCTCCAGACTGTGACC
CTGACAAAGATCACCCTGGAAGCTCAGTTAAAGGAGACTGTGGGAACCCATCTG
TTGGGATTTTCTTTTTTGTCAGTTACATCATCATATCCTTCCTGGTTGTGGTGAAC
ATGTACATCGCGGTCATCCTGGAGAACTTCAGTGTTGCTACTGAAGAAAGTGCAG
AGCCTCTGAGTGAGGATGACTTTGAGATGTTCTATGAGGTTTGGGAGAAGTTTGA
TCCCGATGCGACCCAGTTTATAGAGTTTGCCAAACTTTCTGATTTTGCAGATGCC
CTGGATCCTCCTCTTCTCATAGCAAAACCCAACAAAGTCCAGCTCATTGCCATGG
ATCTGCCCATGGTGAGTGGTGACCGGATCCACTGTCTTGACATCTTATTTGCTTTT
ACAAAGCGTGTTTTGGGTGAGAGTGGAGAGATGGATGCCCTTCGAATACAGATG
GAAGAGCGATTCATGGCATCAAACCCCTCCAAAGTCTCTTATGAGCCCATTACGA
CCACGTTGAAACGCAAACAAGAGGAGGTGTCTGCTATTATTATCCAGAGGGCTT
ACAGACGCTACCTCTTGAAGCAAAAAGTTAAAAAGGTATCAAGTATATACAAGA
AAGACAAAGGCAAAGAATGTGATGGAACACCCATCAAAGAAGATACTCTCATTG
ATAAACTGAATGAGAATTCAACTCCAGAGAAAACCGATATGACGCCTTCCACCA
CGTCTCCACCCTCGTATGATAGTGTGACCAAACCAGAAAAAGAAAAATTTGAAA
AAGACAAATCAGAAAAGGAAGACAAAGGGAAAGATATCAGGGAAAGTAAAAA
GTAAAAAGAAACCAAGAATTTTCCATTTTGTGATCAATTGTTTACAGCCCGTGAT
GGTGATGTGTTTGTGTCAACAGGACTCCCACAGGAGGTCTATGCCAAACTGACTG
TTTTTACAAATGTATACTTAAGGTCAGTGCCTATAACAAGACAGAGACCTCTGGT
CAGCAAACTGGAACTCAGTAAACTGGAGAAATAGTATCGATGGGAGGTTTCTAT
TTTCACAACCAGCTGACACTGCTGAAGAGCAGAGGCGTAATGGCTACTCAGACG
ATAGGAACCAATTTAAAGGGGGGAGGGAAGTTAAATTTTTATGTAAATTCAACA
TGTGACACTTGATAATAGTAATTGTCACCAGTGTTTATGTTTTAACTGCCACACCT
GCCATATTTTTACAAAACGTGTGCTGTGAATTTATCACTTTTCTTTTTAATTCACA
GGTTGTTTACTATTATATGTGACTATTTTTGTAAATGGGTTTGTGTTTGGGGAGAG
GGATTAAAGGGAGGGAATTCTACATTTCTCTATTGTATTGTATAACTGGATATAT
TTTAAATGGAGGCATGCTGCAATTCTCATTCACACATAAAAAAATCACATCACAA
AAGGGAAGAGTTTACTTCTTGTTTCAGGATGTTTTTAGATTTTTGAGGTGCTTAAA
TAGCTATTCGTATTTTTAAGGTGTCTCATCCAGAAAAAATTTAATGTGCCTGTAA
ATGTTCCATAGAATCACAAGCATTAAAGAGTTGTTTTATTTTTACATAACCCATTA
AATGTACATGTATATATGTATATATGTATATGTGCGTGTATATACATATATATGTA
TACACACATGCACACACAGAGATATACACATACCATTACATTGTCATTCACAGTC
CCAGCAGCATGACTATCACATTTTTGATAAGTGTCCTTTGGCATAAAATAAAAAT
ATCCTATCAGTCCTTTCTAAGAAGCCTGAATTGACCAAAAAACATCCCCACCACC
ACTTTATAAAGTTGATTCTGCTTTATCCTGCAGTATTGTTTAGCCATCTTCTGCTCT
TGGTAAGGTTGACATAGTATATGTCAATTTAAAAAATAAAAGTCTGCTTTGTAAA
TAGTAATTTTACCCAGTGGTGCATGTTTGAGCAAACAAAAATGATGATTTAAGCA
CACTACTTATTGCATCAAATATGTACCACAGTAAGTATAGTTTGCAAGCTTTCAA
CAGGTAATATGATGTAATTGGTTCCATTATAGTTTGAAGCTGTCACTGCTGCATG
TTTATCTTGCCTATGCTGCTGTATCTTATTCCTTCCACTGTTCAGAAGTCTAATAT
GGGAAGCCATATATCAGTGGTAAAGTGAAGCAAATTGTTCTACCAAGACCTCAT
TCTTCATGTCATTAAGCAATAGGTTGCAGCAAACAAGGAAGAGCTTCTTGCTTTT
TATTCTTCCAACCTTAATTGAACACTCAATGATGAAAAGCCCGACTGTACAAACA
TGTTGCAAGCTGCTTAAATCTGTTTAAAATATATGGTTAGAGTTTTCTAAGAAAA
TATAAATACTGTAAAAAGTTCATTTTATTTTATTTTTCAGCCTTTTGTACGTAAAA
TGAGAAATTAAAAGTATCTTCAGGTGGATGTCACAGTCACTATTGTTAGTTTCTG
TTCCTAGCACTTTTAAATTGAAGCACTTCACAAAATAAGAAGCAAGGACTAGGA
TGCAGTGTAGGTTTCTGCTTTTTTATTAGTACTGTAAACTTGCACACATTTCAATG
TGAAACAAATCTCAAACTGAGTTCAATGTTTATTTGCTTTCAATAGTAATGCCTTA
TCATTGAAAGAGGCTTAAAGAAAAAAAAAATCAGCTGATACTCTTGGCATTGCTT
GAATCCAATGTTTCCACCTAGTCTTTTTATTCAGTAATCATCAGTCTTTTCCAATG
TTTGTTTACACAGATAGATCTTATTGACCCATATGGCACTAGAACTGTATCAGAT
ATAATATGGGATCCCAGCTTTTTTTCCTCTCCCACAAAACCAGGTAGTGAAGTTA
TATTACCAGTTACAGCAAAATACTTTGTGTTTCACAAGCAACAATAAATGTAGAT
TCTTTATACTGAAGCTATTGACTTGTAGTGTGTTGGTGAAATGCATGCAGGAAAA
TGCTGTTACCATAAAGAACGGTAAACCACATTACAATCAAGCCAAAAGAATAAA
GGTTTCGCTTTTGTTTTTGTATTTAATTGTTGTCTTTGTTTCTATCTTTGAAATGCC
ATTTAAAGGTAGATTTCTATCATGTAAAAATAATCTATCTGAAAAACAAATGTAA
AGAACACACATTAATTACTATAATTCATCTTTCAATTTTTTCATGGAATGGAAGTT
AATTAAGAAGAGTGTATTGGATAACTACTTTAATATTGGCCAAAAAGCTAGATAT
GGCATCAGGTAGACTAGTGGAAAGTTACAAAAATTAATAAAAAATTGACTAACA

What is claimed is:

1. A method for treating a subject with a SCN2A-related disorder caused by a gain-of-function SCN2A mutation, the method comprising:
determining whether the subject has had an early onset seizure; and
administering a SCN2A inhibitor to the subject if the subject has had an early onset seizure,
wherein the method does not comprise experimentally verifying that the subject's SCN2A mutation as a gain-of-function mutation or confirming that the subject's SCN2A mutation has previously been experimentally verified as a gain-of-function mutation prior to administering the SCN2A inhibitor.

2. The method of claim 1, wherein the early onset seizure occurred within the first three months, the first two and a half months, the first two months, the first month and a half, the first month, the first four weeks, the first three weeks, the first two weeks, the first week, the first day of the subject's life, or while the subject was in utero.

3. The method of claim 1 or claim 2, further comprising determining that the subject carries a SCN2A mutation prior to administering the SCN2A inhibitor without experimentally verifying that the SCN2A mutation is a gain-of-function mutation or confirming that the SCN2A mutation has previously been experimentally verified as a gain-of-function mutation prior to administering the SCN2A inhibitor.

4. The method of any one of claims 1-3, wherein the SCN2A mutation is A263V, E430A, R1882Q, G879R, G1593R, K1502N, V1601L, G211D, S1780I, D343H, or combinations of the foregoing.

5. The method of any one of claims 1-4, wherein the subject is less than 18 years old.

6. The method of any one of claims 1-5, wherein the SCN2A-related disorder caused by a gain-of-function SCN2A mutation is Ohtahara syndrome, epilepsy of infancy with migrating focal seizures, or early onset epileptic encephalopathy.

7. The method of any one of claims 1-6, wherein the SCN2A inhibitor is administered intrathecally, intramedullarly, intracerebroventricularly, or parenterally.

8. The method of claim 7, wherein the parenteral administration is subcutaneous, intravenous, or intramuscular injection or infusion.

9. The method of any one of claims 1-8, wherein the SCN2A inhibitor is a CRISPR-Cas repressor, a SCN2A channel blocker, an antibody, or a nucleic acid inhibitor molecule, such as an antisense oligonucleotide or an siRNA.

10. The method of claim 9, wherein the antisense oligonucleotide comprises a single-stranded oligonucleotide that is 10-80 nucleosides in length and comprises a nucleobase sequence comprising a portion of 10 contiguous nucleobases having at least 80% complementary to an equal length portion of a target region of a pre-mRNA transcript or an mRNA transcript of a human SCN2A gene, in an amount and for a duration sufficient to treat the SCN2A-related disorder caused by a gain-of-function SCN2A mutation.

11. The method of claim 10, wherein the method decreases expression of the human SCN2A gene.

12. The method of claim 10 or claim 11, wherein the oligonucleotide comprises, consists essentially of, or consists of a nucleobase sequence complementary to a portion of SCN2A mRNA that encodes the amino acid sequence of SEQ ID NO: 1.

13. The method of any one of claims 10-12, wherein the oligonucleotide comprises one or more modified sugars, one or more modified internucleoside linkages, and/or one or more modified nucleobases.

14. The method of claim 13, wherein the oligonucleotide comprises one or more modified sugars.

15. The method of claim 14, wherein each of the one or more modified sugars is independently selected from the group consisting of a bicyclic sugar, a 2′-O-methoxyethyl (2MOE) modified sugar, a 2′-O-methyl (2-OMe) modified sugar, a 2′-methoxy modified sugar, a 2′-Fluoro modified sugar, a 2′-O-alkyl modified sugar, a constrained ethyl (cEt) modified sugar, a locked sugar, and an unlocked sugar.

16. The method of claim 15, wherein each nucleoside of the oligonucleotide comprises a 2MOE modified sugar.

17. The method of any one of claims 13-16, wherein the oligonucleotide comprises one or more modified internucleoside linkages.

18. The method of claim 17, wherein one or more of the modified internucleoside linkages comprises a modified phosphate.

19. The method of claim 18, wherein each of the modified phosphates is independently selected from the group consisting of a phosphorothioate, a phosphorodithioate, a phosphoramidate, a phosphorodiamidate, a thiophosphoramidate, a thiophosphorodiamidate, a methyl phosphonate, a phosphoromorpholidate, and a phosphoropiperazidate.

20. The method of claim 19, wherein each internucleoside linkage of the oligonucleotide is a phosphorothioate internucleoside linkage.

21. The method of claim 20, wherein the each phosphorothioate internucleoside linkage is a phosphorodiamidate morpholino internucleoside linkage.

22. The method of any one of claims 13-21, wherein the oligonucleotide comprises one or more modified nucleobases.

23. The method of claim 22, wherein the modified nucleobase is selected from the group consisting of 5-methylcytosine, 5-hydroxymethyl cytosine, xanthine, hypoxanthine, 2-aminoadenine, 6-methyladenine, 6-methylguanine, 2-propyladenine, 2-propylguanine, 2-thiouracil, 2-thiothymine, 2-thiocytosine, 5-halouracil, 5-halocytosine, 5-propynyluracil, 5-propynylcytosine, 6-azouracil, 6-azocytosine, 6-azothymine, 5-uracil (pseudouracil), 4-thiouracil, 8-haloadenine, 8-aminoadenine, 8-thioladenine, 8-thioalkyladenine, 8-hydroxyladenine, 8-haloguanine, 8-aminoguanine, 8-thiolguanine, 8-thioalkylguanine, 8-hydroxylguanine, 5-bromouracil, 5-trifluoromethyluracil, 5-bromocytosine, 5-trifluoromethylcytosine, 7-methylguanine, 7-methyladenine, 2-fluoroadenine, 8-azaguanine, 8-azaadenine, 7-deazaguanine, 7-deazaadenine, 3-deazaguanine, and 3-deazaadenine.

24. The method of claim 23, wherein the modified nucleobase is a 5-methylcytosine.

25. The method of claim 24, wherein each cytosine in the oligonucleotide is a 5-methylcytosine.

26. The method of any one of claims 13-25, wherein the modified oligonucleotide comprises: a gap segment consisting of linked deoxynucleosides; a 5′ wing segment consisting of linked nucleosides; and a 3′ wing segment consisting of linked nucleosides; wherein the gap segment is positioned immediately adjacent to and between the 5′ wing segment and the 3′ wing segment and wherein each nucleoside of each wing segment comprises a modified sugar.

27. The method of any one of claims 10-26, wherein the oligonucleotide consists of 12 to 40 nucleobases.

28. The method of claim 27, wherein the oligonucleotide consists of 16 to 30 nucleobases.

29. The method of any one of claims 1-28, wherein the method comprises inhibiting the expression of SCN2A in neuronal cells in the subject.

30. The method of any one of claims 10-29, wherein the oligonucleotide is selective for SCN2A pre-mRNA or mRNA over SCN1A pre-mRNA or mRNA.

31. A composition comprising an SCN2A inhibitor for use in the methods of any one of claims 1-30.

32. Use of an SCN2A inhibitor for treating a subject with a SCN2A-related disorder caused by a gain-of-function SCN2A mutation according to the method of any one of claims 1-30.

33. A method for treating a subject with a SCN2A-related disorder caused by a loss-of-function SCN2A mutation, the method comprising:
determining whether the subject has had an early onset seizure; and
administering a SCN2A enhancer to the subject if the subject has not had an early onset seizure,
wherein the SCN2A enhancer is administered as a second line therapy or is co-administered with a first line therapy if the subject has infantile spasms, and wherein the method does not comprise experimentally verifying the subject's SCN2A mutation as a loss-of-function mutation or confirming that the subject's SCN2A mutation has previously been experimentally verified as a loss-of-function mutation prior to administering the SCN2A enhancer.

34. The method of claim 33, wherein the first line therapy is steroids, adrenocorticotropic hormone (ACTH), or vigabatrin.

35. The method of claim 33 or claim 34, wherein the subject had no seizure within the first six months, within the first five months, within the first four months, within the first three months, within the first two and a half months, within the first two months, within the first month, or within the first four weeks of the subject's life.

36. The method of any one of claims 33-35, further comprising determining that the subject carries a SCN2A mutation prior to administering the SCN2A enhancer without experimentally verifying that the SCN2A mutation is a loss-of-function mutation or confirming that the SCN2A mutation has previously been experimentally verified as a loss-of-function mutation prior to administering the SCN2A enhancer.

37. The method of any one of claims 33-36, wherein the SCN2A mutation comprises M951R, F1375V, R853Q, A1773T, R571H, K1422E, D195G, Y428, K507E, Y1771H, S1758R, or combinations of the foregoing.

38. The method of any one of claims 33-37, wherein the subject is less than 18 years old.

39. The method of any one of claims 33-38, wherein the SCN2A-related disorder caused by a loss-of-function SCN2A mutation is autism spectrum disorder, benign familial neonatal/infantile seizures, infantile spasms, Ohtahara syndrome, late seizure onset epileptic encephalopathy, or epilepsy of infancy with migrating focal seizures.

40. The method of any one of claims 33-39, wherein the SCN2A enhancer is administered intrathecally, intramedullarly, intracerebroventricularly, or parenterally.

41. The method of claim 40, wherein the parenteral administration is subcutaneous, intravenous, or intramuscular injection or infusion.

42. The method of any one of claims 33-41, wherein the SCN2A enhancer is an antisense oligonucleotide, a CRISPR-Cas enhancer, an antibody, or a SCN2A activator.

43. The method of claim 42, wherein the antisense oligonucleotide comprises a single-stranded oligonucleotide that is 10-80 nucleosides in length and comprises a nucleobase sequence comprising a portion of 10 contiguous nucleobases having at least 80% complementary to an equal length portion of a target region of a pre-mRNA transcript or an mRNA transcript of a human SCN2A gene, in an amount and for a duration sufficient to treat the SCN2A-related disorder caused by a loss-of-function SCN2A mutation.

44. The method of claim 43, wherein the method increases expression of the human SCN2A gene.

45. The method of claim 43 or claim 44, wherein the oligonucleotide comprises, consists essentially of, or consists of a nucleobase sequence complementary to a portion of SCN2A mRNA that encodes the amino acid sequence of SEQ ID NO: 1.

46. The method of any one of claims 43-45, wherein the oligonucleotide comprises one or more modified sugars, one or more modified internucleoside linkages, and/or one or more modified nucleobases.

47. The method of claim 46, wherein the oligonucleotide comprises one or more modified sugars.

48. The method of claim 47, wherein each of the one or more modified sugars is independently selected from the group consisting of a bicyclic sugar, a 2′-O-methoxyethyl (2MOE) modified sugar, a 2′-O-methyl (2-OMe) modified sugar, a 2′-methoxy modified sugar, a 2′-Fluoro modified sugar, a 2′-O-alkyl modified sugar, a constrained ethyl (cEt) modified sugar, a locked sugar, and an unlocked sugar.

49. The method of claim 48, wherein each nucleoside of the oligonucleotide comprises a 2MOE modified sugar.

50. The method of any one of claims 46-49, wherein the oligonucleotide comprises one or more modified internucleoside linkages.

51. The method of claim 50, wherein one or more of the modified internucleoside linkages comprises a modified phosphate.

52. The method of claim 51, wherein each of the modified phosphates is independently selected from the group consisting of a phosphorothioate, a phosphorodithioate, a phosphoramidate, a phosphorodiamidate, a thiophosphoramidate, a thiophosphorodiamidate, a methyl phosphonate, a phosphoromorpholidate, and a phosphoropiperazidate.

53. The method of claim 52, wherein each of the internucleoside linkages of the oligonucleotide is a phosphorothioate internucleoside linkage.

54. The method of claim 53, wherein each of the phosphorothioate internucleoside linkages is a phosphorodiamidate morpholino internucleoside linkage.

55. The method of any one of claims 43-54, wherein the oligonucleotide comprises one or more modified nucleobases.

56. The method of claim 55, wherein the modified nucleobase is selected from the group consisting of 5-methylcytosine, 5-hydroxymethyl cytosine, xanthine, hypoxanthine, 2-aminoadenine, 6-methyladenine, 6-methylguanine, 2-propyladenine, 2-propylguanine, 2-thiouracil, 2-thiothymine, 2-thiocytosine, 5-halouracil, 5-halocytosine, 5-propynyluracil, 5-propynylcytosine, 6-azouracil, 6-azocytosine, 6-azothymine, 5-uracil (pseudouracil), 4-thiouracil, 8-haloadenine, 8-aminoadenine, 8-thioladenine, 8-thioalkyladenine, 8-hydroxyladenine, 8-haloguanine, 8-aminoguanine, 8-thiolguanine, 8-thioalkylguanine, 8-hydroxylguanine, 5-bromouracil, 5-trifluoromethyluracil, 5-bromocytosine, 5-trifluoromethylcytosine, 7-methylguanine, 7-methyladenine, 2-fluoroadenine, 8-azaguanine, 8-azaadenine, 7-deazaguanine, 7-deazaadenine, 3-deazaguanine, and 3-deazaadenine.

57. The method of claim 56, wherein the modified nucleobase is a 5-methylcytosine.

58. The method of claim 57, wherein each cytosine of the oligonucleotide is a 5-methylcytosine.

59. The method of any one of claims 43-58, wherein the modified oligonucleotide comprises: a gap segment consisting of linked deoxynucleosides; a 5′ wing segment consisting of linked nucleosides; and a 3′ wing segment consisting of linked nucleosides; wherein the gap segment is positioned immediately adjacent to and between the 5′ wing segment and the 3′ wing segment and wherein each nucleoside of each wing segment comprises a modified sugar.

60. The method of any one of claims 43-59, wherein the oligonucleotide consists of 12 to 40 nucleobases.

61. The method of claim 60, wherein the oligonucleotide consists of 16 to 30 nucleobases.

62. The method of any one of claims 33-61, wherein the method comprises increasing the expression of SCN2A in neuronal cells in the subject.

63. The method of any one of claims 43-62, wherein the oligonucleotide is selective for SCN2A pre-mRNA or mRNA over SCN1A pre-mRNA or mRNA.

64. A composition comprising an SCN2A enhancer for use in the methods of any one of claims 33-63.

65. Use of an SCN2A enhancer for treating a subject with a SCN2A-related disorder caused by a loss-of-function SCN2A mutation according to the method of any one of claims 33-63.
